(19) United States
(12) Patent Application Publication
(10) Pub. No.: US 2018/0200194 A1
(43) Pub. Date: Jul. 19, 2018

(54) DECOY NANOPARTICLES TO DISRUPT CANCER CELL-STROMAL CELL NETWORKS

(71) Applicant: The John Hopkins University, Baltimore, MD (US)

(72) Inventors: Zaver M. Bhujwalla, Baltimore, MD (US); Jiefu Jin, Lutherville Timonium, MD (US); Sridhar Nimmagadda, Baltimore, MD (US); Balaji Krishnamachary, Baltimore, MD (US)

(21) Appl. No.: 15/752,845

(22) PCT Filed: Aug. 12, 2016
(86) PCT No.: PCT/US16/46692

§ 371 Date: Feb. 14, 2018

Related U.S. Application Data
(60) Provisional application No. 62/205,225, filed on Aug. 14, 2015.

(51) Int. Cl. A61K 9/51; A61K 9/00; A61K 9/14; A61K 38/17; A61P 35/00

(52) U.S. Cl. CPC A61K 9/51; A61K 9/0019; A61P 35/00; A61K 38/1793; A61K 9/146

(57) ABSTRACT

The present invention relates to compositions and methods for disrupting cancer stromal cell networks using synthetic nanoparticles coated with plasma membranes.

### RELATED APPLICATIONS

This application is an International Patent Application which claims the benefit of priority under 35 U.S.C. § 119(e) to U.S. Provisional Application No. 62/205,225, filed on Aug. 14, 2015, which is incorporated herein by reference in its entirety.

### STATEMENT REGARDING FEDERALLY SPONSORED RESEARCH OR DEVELOPMENT

This invention was made with government support under R21 CA 198243 and P50 CA103175 awarded by the National Institutes of Health (NIH). The government has certain rights in the invention.

### BACKGROUND OF THE INVENTION

Prior to the invention described herein, metastatic cancer continued to be a major cause of mortality from breast and other cancers. As such, there is an unmet need for strategies aimed at treating cancer.

### SUMMARY OF THE INVENTION

The present invention is based, at least in part, upon the development of nanoparticles coated with plasma membranes derived from cancer cells. Such plasma membrane-camouflaged nanoparticles retain the membrane-associated components (lipids, proteins, and carbohydrates) in a native-like state within the cell membranes after isolation and translocation to the surface of nanoparticles where all components present in the right-side-out orientation. This biomimetic strategy provides the advantage of replicating the complex surface of the cancer cell plasma membrane profile on the nanoparticle surface. The nanoparticles described herein are used as decoys to misdirect cancer cell signaling or as vaccines to activate the immune response to a subject's cancer. Furthermore, the nanoparticles have the capacity to carry therapeutic cargoes or imaging reporters for cell-specific delivery application such as treating or detecting the cancer, respectively.

The compositions, e.g., nanoparticles, and methods described herein are useful as anti-cancer agents to inhibit tumor growth in a subject. The compositions of the present invention also play roles as cancer vaccines and biomimetic delivery systems. The subject is preferably a mammal in need of such treatment, e.g., a subject that has been diagnosed with cancer or a predisposition thereto. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

Provided herein are compositions comprising a nanoparticle, wherein the nanoparticle surface is encapsulated with one or more plasma membrane-associated components, and wherein the plasma membrane is derived from a cancer cell. An exemplary cancer cell comprises a breast cancer cell. For example, the plasma membrane-associated component comprises a lipid, a protein, or a carbohydrate. For example, the plasma membrane-associated component comprises roughly 75% lipids (e.g., phospholipids), 20% proteins (e.g., integral and peripheral membrane proteins), and 5% carbohydrates (e.g., carbohydrates in glycolipids and glycoproteins). Preferably, the plasma membrane-associated component is retained in a native conformation within the plasma membrane on the surface of the nanoparticle, i.e., the plasma membrane-associated component is present on the surface of the nanoparticle in the same or similar conformation as it was present on the surface of the cancer cell. In some cases, the plasma membrane-associated component is present in the right-side-out orientation on the surface of the nanoparticle.

In some cases, the plasma membrane comprises a bilayer. Optionally, the plasma membrane is about 1 nm-about 10 nm thick, e.g., about 1 nm, about 2 nm, about 3 nm, about 4 nm, about 5 nm, about 6 nm, about 7 nm, about 8 nm, about 9 nm, or about 10 nm thick. Optionally, the plasma membrane is about 5 nm thick.

Optionally, the nanoparticle is negatively charged. Suitable nanoparticles include those that are about 1 nm to about 100 nm in size, e.g., 10 nm, 20 nm, 30 nm, 40 nm, 50 nm, 60 nm, 70 nm, 80, nm, 90 nm, or 100 nm in size. For example, the nanoparticle comprises a polymeric nanoparticle, e.g., a polylactic-co-glycolic acid (PLGA) polymeric nanoparticle.

In some cases, the compositions further comprise a detectable label. For example, the label further comprises a fluorescent dye, a contrast agent, or a radioisotope. Suitable fluorescent dyes include a far red dye, e.g., a 1,1′-dioctadecyl-3,3,3′,3′-tetramethylindodicarbocyanine, 4-chlorobenzenesulfonate salt.

Optionally, the composition further comprises a targeting molecule. For example, the targeting molecule is selected from the group comprising of an amino acid, an antibody, a protein, an enzyme, a peptide, an oligopeptide, a nucleic acid, a peptide nucleic acid, a lipid, a fatty acid, a glycerolipid, a glycolipid, a glycoprotein, a polysaccharide, a receptor, a ligand, a hormone, a steroid, an antibiotic, and a chemotherapeutic. For example, the antibody comprises anti-smooth muscle actin antibody (α-SMA antibody) or anti-fibroblast activation protein alpha (anti-FAP-α antibody).

In some cases, the plasma membrane comprises CXCR4. For example, the CXCR4 on the plasma membrane binds to CXCL12 released by fibroblasts. Other exemplary cancer receptors/antigens include but are not limited to: CEA (carcinoembryonic antigen), HER2 (human epidermal growth factor receptor 2), CD44 (cluster of differentiation 44) and PSMA (prostate-specific membrane antigen).

In one aspect, the methods described herein involve administering (e.g., injecting) approximately 100 μl of a 0.1 mg/ml nanoparticle solution containing approximately 1.5×10¹⁰ nanoparticles. For example, about 1 μl, about 10 μl, about 25 μl, 50 μl, about 75 μl, about 100 μl, about 150 μl, about 200 μl, about 300 μl, about 400 μl, about 500 μl, about 600 μl, about 700 μl, about 800 μl, about 900 μl, or about 1 ml of nanoparticle solution is administered. In some cases, the nanoparticle solution is administered at a concentration of about 0.01 mg/ml, about 0.02 mg/ml, about 0.03 mg/ml, about 0.04 mg/ml, about 0.05 mg/ml, about 0.06 mg/ml, about 0.07 mg/ml, about 0.08 mg/ml, about 0.09 mg/ml, about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, or about 1 mg/ml. For example, about 1.5×10⁶, about 1.5×10⁷, about 1.5×10⁸, about 1.5×10⁹, about 1.5×10¹⁰, about 1.5×10¹¹, about 1.5×10¹², about 1.5×10¹³, or about 1.5×10¹⁴ nanoparticles are administered.

Methods of treating cancer are carried out by isolating a cancer cell from a subject; administering to the subject a composition comprising a nanoparticle, wherein the nanoparticle surface is encapsulated with one or more plasma membrane-associated components, and wherein the plasma membrane is derived from the cancer cell; and activating an immune response against the cancer cell in the subject, thereby treating the cancer.

Exemplary cancers are selected from the group consisting of breast cancer, skin cancer, lung cancer, brain cancer, pancreatic cancer, esophageal cancer, stomach cancer, liver cancer, kidney cancer, colorectal cancer, intestinal cancer, bladder cancer, prostate cancer, ovarian cancer, uterine cancer, testicular cancer, sarcoma, lymphoma, leukemia, retinoblastoma, oral cancer, bone cancer, neoplasia, dysplasia, and glioma.

In some cases, the composition further comprises a drug or pharmaceutical composition. For example, the drug or pharmaceutical composition comprises a chemotherapeutic composition.

Also provided are methods of disrupting cancer cell-stromal cell signaling in a subject comprising: isolating a cancer cell from a subject; administering to the subject a composition comprising a nanoparticle, wherein the nanoparticle surface is encapsulated with one or more plasma membrane-associated components, and wherein the plasma membrane is derived from the cancer cell, thereby disrupting cancer cell-stromal cell signaling in the subject.

Also provided are methods of detecting a cancer cell-stromal cell interaction comprising:

isolating a cancer cell from a subject; administering to the subject a composition comprising a nanoparticle, wherein the nanoparticle surface is encapsulated with one or more plasma membrane-associated components, and wherein the plasma membrane is derived from the cancer cell, wherein the composition further comprises a detectable label; and identifying the detectable label, thereby detecting a cancer cell-stromal cell interaction. For example, the label comprises a fluorescent dye, a contrast agent, or a radioisotope. In some cases, the fluorescent dye comprises a far red dye, e.g., a 1,1′-dioctadecyl-3,3,3′,3′-tetramethylindodicarbocyanine, 4-chlorobenzenesulfonate salt.

Also provided are methods of preparing a composition comprising a nanoparticle, wherein the nanoparticle surface is encapsulated with one or more plasma membrane-associated components, and wherein the plasma membrane is derived from a cancer cell are carried out by isolating a cancer cell; fractionating the cancer cell into one or more plasma membrane-derived vesicles; synthesizing polymeric nanoparticles; and fusing the plasma membrane-derived vesicle with the nanoparticle, thereby preparing a composition comprising a nanoparticle. For example, fractionating the cancer cell into one or more plasma membrane-derived vesicles comprises sequentially homogenizing and gradient-density centrifuging the cancer cell. In one aspect, fusing the plasma membrane-derived vesicle with the nanoparticle comprises mixing the plasma membrane-derived vesicle with the nanoparticle and physically extruding the mixture through a porous membrane. Optionally, the porous membrane comprises a 100 nm polycarbonate porous membrane.

In some cases, the methods described herein are used in conjunction with one or more agents or a combination of additional agents, e.g., an anti-cancer agent. Suitable agents include current pharmaceutical and/or surgical therapies for an intended application, such as, for example, cancer. For example, the methods described herein can be used in conjunction with one or more chemotherapeutic or anti-neoplastic agents. In some cases, the additional chemotherapeutic agent is radiotherapy. In some cases, the chemotherapeutic agent is a cell death-inducing agent.

The term “antineoplastic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

In some cases, a composition of the invention is administered orally or systemically. Other modes of administration include rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy may be most appropriate for pulmonary vascular diseases (e.g., pulmonary hypertension).

Pharmaceutical compositions may be assembled into kits or pharmaceutical systems comprising the nanoparticles described herein. Kits or pharmaceutical systems according to this aspect of the invention comprise a carrier means, such as a box, carton, tube, having in close confinement therein one or more container means, such as vials, tubes, ampoules, bottles, syringes, or bags. The kits or pharmaceutical systems of the invention may also comprise associated instructions for using the kit.

### Definitions

By “agent” is meant any small compound, antibody, nucleic acid molecule, or polypeptide, or fragments thereof.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “ameliorate” is meant decrease, suppress, attenuate, diminish, arrest, or stabilize the development or progression of a disease.

By “cancer” (also called neoplasia, dysplasia, malignant tumor, and/or malignant neoplasia) is meant a group of diseases involving abnormal cell growth with the potential to invade or spread to other parts of the body. Not all tumors are cancerous; benign tumors do not spread to other parts of the body. There are over 100 different known cancers that affect humans.

The transitional term “comprising,” which is synonymous with “including,” “containing,” or “characterized by,” is inclusive or open-ended and does not exclude additional, unrecited elements or method steps. By contrast, the transitional phrase “consisting of excludes any element, step, or ingredient not specified in the claim. The transitional phrase” consisting essentially of limits the scope of a claim to the specified materials or steps “and those that do not materially affect the basic and novel characteristic(s)” of the claimed invention.

“Detect” refers to identifying the presence, absence or amount of the analyte to be detected.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to reduce or prevent cancer or cancer metastasis in a mammal. Ultimately, the attending physician or veterinarian decides the appropriate amount and dosage regimen.

By “fibroblast” is meant a type of cell that synthesizes the extracellular matrix and collagen, the structural framework (stroma) for animal tissues, and plays a critical role in wound healing. Fibroblasts are the most common cells of connective tissue in animals.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, 100, 200, 300, 400, 500, 600, 700, 800, 900, or 1000 nucleotides or amino acids.

By “immunotherapy” is meant the “treatment of disease by inducing, enhancing, or suppressing an immune response” Immunotherapies designed to elicit or amplify an immune response are classified as activation immunotherapies, while immunotherapies that reduce or suppress are classified as suppression immunotherapies.

The terms “isolated,” “purified,” or “biologically pure” refer to material that is free to varying degrees from components which normally accompany it as found in its native state.

“Isolate” denotes a degree of separation from original source or surroundings. “Purify” denotes a degree of separation that is higher than isolation.

By “marker” is meant any protein or polynucleotide having an alteration in expression level or activity that is associated with a disease or disorder.

By “modulate” is meant alter (increase or decrease). Such alterations are detected by standard art known methods such as those described herein.

By “nanoparticle” is meant particles between 1 and 100 nanometers in size. In nanotechnology, a particle is defined as a small object that behaves as a whole unit with respect to its transport and properties. Particles are further classified according to diameter. Ultrafine particles are the same as nanoparticles and between 1 and 100 nanometers in size, fine particles are sized between 100 and 2,500 nanometers, and coarse particles cover a range between 2,500 and 10,000 nanometers.

By “plasma membrane” (also known as the cell membrane or cytoplasmic membrane) is meant a biological membrane that separates the interior of all cells from the outside environment. The plasma membrane is selectively permeable to ions and organic molecules and controls the movement of substances in and out of cells. The basic function of the plasma membrane is to protect the cell from its surroundings. It consists of the phospholipid bilayer with embedded proteins. Plasma membranes are involved in a variety of cellular processes such as cell adhesion, ion conductivity and cell signaling and serve as the attachment surface for several extracellular structures, including the cell wall, glycocalyx, and intracellular cytoskeleton. Plasma membranes can be artificially reassembled.

A “purified” or “biologically pure” nucleic acid or protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 1%, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%.

By “reference” is meant a standard or control condition.

A “reference sequence” is a defined sequence used as a basis for sequence comparison or a gene expression comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 40 nucleotides, preferably at least about 60 nucleotides, more preferably at least about 75 nucleotides, and even more preferably about 100 nucleotides or about 300 or about 500 nucleotides or any integer thereabout or there between.

As used herein, “obtaining” as in “obtaining an agent” includes synthesizing, purchasing, or otherwise acquiring the agent.

By “stromal cell” is meant connective tissue cells of any organ, for example in the uterine mucosa (endometrium), prostate, bone marrow, and the ovary. They are cells that support the function of the parenchymal cells of that organ. Fibroblasts and pericytes are among the most common types of stromal cells.

By “subject” is meant a mammal, including, but not limited to, a human or non-human mammal, such as a bovine, equine, canine, ovine, or feline. The subject is preferably a mammal in need of treatment, e.g., a subject that has been diagnosed with cancer or a predisposition thereto. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Unless specifically stated or obvious from context, as used herein, the term “or” is understood to be inclusive. Unless specifically stated or obvious from context, as used herein, the terms “a”, “an”, and “the” are understood to be singular or μlural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

A “therapeutically effective amount” is an amount sufficient to effect beneficial or desired results, including clinical results. An effective amount can be administered in one or more administrations.

Any compositions or methods provided herein can be combined with one or more of any of the other compositions and methods provided herein.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1 depicts an overlay of collagen 1 (Col1) fibers in green with human fibroblasts in red in a highly metastatic TNBC MDA-MB-231 tumor (left) and a poorly metastatic TNBC tumor (right). Human fibroblasts were injected intravenously. Fewer fibroblasts are evident in the poorly metastatic tumor with fewer Col1 fibers.

FIG. 2 depicts the correlation between individual nodule pixels (reflecting nodule size) and strongly positive pixels (reflecting number of activated fibroblasts in the corresponding nodule). A significant correlation was observed supporting the role of activated fibroblasts in the formation of metastasis.

FIG. 3 depicts a 3D visualization of Col1 fibers obtained by second harmonic generation (SHG) microscopy in highly metastatic parental TNBC MDA-MB-231 tumors (left) and poorly metastatic COX-2 reduced tumors (right). The field of view (FOV) image size was 334.91×334.91×15 μm³ with a voxel size of 0.66×0.66×1 μm³. Significantly fewer fibers are observed in the poorly metastatic tumor.

FIG. 4 depicts representative images of α-SMA immunostained sections obtained from highly metastatic parental TNBC MDA-MB-231 tumors (left) and poorly metastatic COX-2 reduced tumors (right). Significantly higher CAFs are observed in the metastatic tumor as evident from the high density of brown staining.

FIG. 5 depicts α-SMA immunostaining of lung nodules detecting increased number of CAFs (white arrows) in metastatic nodules from highly metastatic MDA-MB-231 TNBC (left) and hardly any CAFs in lung nodules from poorly metastatic COX-2 downregulated MDA-MB-231 TNBC (right).

FIG. 6 depicts an overlay of Col1 fibers in green with hematoxylin and eosin (left-lung nodule from highly metastatic tumor MDAMB-231 TNBC, right-lung nodule from poorly metastatic COX-2 downregulated MDA-MB-231 TNBC). Col1 fibers were imaged using SHG microscopy. Fewer Col1 fibers are evident in the nodule from the poorly metastatic tumor.

FIG. 7A shows TEM micrographs of PLGA NPs, U87MG-CXCR4 cell membrane-derived vesicles and membrane-coated decoy NPs. FIG. 7B shows size intensity curves of PLGA NPs (PDI 0.28) and U87MG-CXCR4 (PDI 0.271) cell membrane-derived vesicles measured by DLS.

FIGS. 8A and 8B depict analysis of protein content. FIG. 8A shows Western blot analysis of U87 and U87-CXCR4 cells using post nuclear supernatant (PNS), crude membrane (CM) and membrane fraction (MF) with antibodies against membrane specific protein markers (pan-cadherin and Na+/K+-ATPase), CXCR4 and cytosol marker (GAPDH). FIG. 8B shows fluorescence images of membrane fractions of U87 and U87-CXCR4 cells upon staining with PE-conjugated antihuman CXCR4 antibody (upper panel) and PE-conjugated mouse IgG isotype control (lower panel).

FIG. 9 depicts a schematic illustration of decoy NPs made by coating cancer cell membrane vesicles on PLGA NPs.

FIG. 10 depicts a proposed migration of the decoy NPs to CXCL12 gradient of the CAFs. The decoy NPs enriched with CXCR4 receptors on the surface act as a nanosponge for CXCL12.

FIG. 11 depicts a schematic of decoy NP with CAF binding FAP-α antibody.

FIG. 12 is a schematic illustration of the preparation of cancer cell plasma membrane fraction coated PLGA NPs (CCMF-PLGA NPs). Cancer cell derived plasma membrane fractions (CCMFs) were derived from their source cells through a series of homogenization, differential centrifugation, and sucrose density gradient centrifugation treatments. CCMFs together with its associated proteins are translocated to PLGA NPs through extrusion processes.

FIG. 13A, FIG. 13B, FIG. 13C, and FIG. 13D is a photograph of an immunoblot, a series of photomicrographs, a line graph and a bar chart showing the characterization of PLGA NPs, U87-CXCR4 MFs, and U87-CXCR4 MFs+PLGA NPs. FIG. 13A is a western blotting analysis by probing plasma membrane-specific marker (Na+/K+-ATPase), endoplasmic reticulum marker (GRP78), mitochondrial maker (ATP5a), and cytosol marker (GAPDH). Notations: Lys (cell lysate), PNS (post nuclear supernatant), Mito (mitochondria fraction), CM (crude membrane), and MF (membrane fraction). FIG. 13B is a series of photomicrographs showing representative TEM images of PLGA NPs, U87-CXCR4 MFs, and U87-CXCR4 MFs+PLGA NPs with insets showing high magnification images. Scale bars in the inserts are 100 nm, 500 nm, and 20 nm, respectively. FIG. 13C and FIG. 13D show number distribution curves and zeta-potential values, respectively, of PLGA NPs, and U87-CXCR4 MFs, and U87-CXCR4 MFs+PLGA NPs measured by DLS.

FIG. 14 A-FIG. 14B is a series of photomicrographs and a series of line graphs showing that U87-CXCR4 MFs and U87-CXCR4 MFs+PLGA NPs expose their surface proteins in the right-side-out manner FIG. 14A is a series of confocal microscopic images of MFs and MFs+PLGA NPs stained with PE-conjugated anti-human CXCR4 antibody (upper panel, only recognizing extracellular CXCR4 epitope) and PE-conjugated isotype IgG2a control. FIG. 14B is a series of graphs showing flow cytometric analysis on U87-CXCR4 cells, U87-CXCR4 MFs and U87-CXCR4 MFs+PLGA NPs after staining with PE-conjugated anti-human CXCR4 antibody. U87 compartments and PE-conjugated isotype IgG2a were used as controls.

FIG. 15A, FIG. 15B, FIG. 15C, and FIG. 15D are a series of bar charts and a series of photomicrographs showing the results of a functional study of CCMFs and CCMFs+PLGA NPs. FIG. 15A is a bar chart showing the percent cancer cells migrating towards 10 nM of CXCL12 in the presence or absence of CCMFs. Values are normalized to number of cancer cells migrating towards HMFs without MFs. FIG. 15B is a bar chart showing the percent cancer cells migrating towards 1% FBS in the presence or absence of CCMFs. Values are normalized to number of cancer cells migrating towards HMFs without MFs. FIG. 15C is a bar chart showing the percent cancer cells migrating towards HMFs in the presence or absence of pre-incubation of CCMFs or CCMF+PLGA NPs. Values are normalized to number of cancer cells migrating towards 10 nM CXCL12, 1% FBS, or HMFs, respectively. *P<0.05 for CCMFs and CCMF+PLGA NPs groups compared to the HMF groups using Student's t test. FIG. 15D is a series of representative bright-field images of the migrated cancer cells corresponding to the values as shown in FIG. 15C.

FIG. 16 is a series of photographs showing NIR mouse images before, 24 h and 48 h after injection of a suspension of 0.1 mg of cancer cell membrane vesicles in 0.05 ml PBS in the foot pad, and near the axilla. The sciatic lymph node (arrow and inset) is clearly detected by NIR imaging by 24 h. The strong signal in the axilla did not allow identification of proximal axillary lymph nodes.

### DETAILED DESCRIPTION OF THE INVENTION

The present invention is based, at least in part, upon the development of nanoparticles coated with plasma membrane derived from cancer cells. These plasma membrane-coated nanoparticles retain the membrane-associated components (lipids, proteins, and carbohydrates) in a native-like state within the cell membranes after isolation and translocation to the surface of nanoparticles where all components present in the right-side-out orientation. In some embodiments, the plasma membrane coated nanoparticles replicate the complex surface of the cancer cell plasma membrane on the nanoparticle surface. This further allows the nanoparticles to act: (1) as decoys to misdirect cancer signaling or (2) as vaccines to activate the immune response to a subject's cancer. In some embodiments, the nanoparticles are loaded with therapeutic cargoes or imaging reporters for treating or detecting cancer, respectively. In some embodiments, the compositions and methods of the present invention are used to treat cancer. In some cases, the compositions and methods of the present invention are used to treat breast cancer.

Stromal cells such as cancer associated fibroblasts (CAFs) mediate many of the aggressive characteristics of cancer (Horimoto Y, Polanska U M, Takahashi Y, Orimo A. Emerging roles of the tumor-associated stroma in promoting tumor metastasis. Cell Adh Migr. 2012; 6(3):193-202), but have an ever-replenishing supply that is largely left intact by current therapeutic strategies (Eyden B. The myofibroblast: phenotypic characterization as a prerequisite to understanding its functions in translational medicine. J Cell Mol Med. 2008; 12(1):22-37). Because of their important functional roles, destroying stromal cells that assist cancer cells is not a viable solution. Instead, as described in detail below, disrupting communications between cancer cells and stromal cells is a useful strategy. The present invention provides nanoparticles (NPs) that attach to CAFs and disrupt the CXCL12-CXCR4 axis, which has a wide spectrum of roles in facilitating breast cancer invasion and metastasis through breast cancer-CAF signaling.

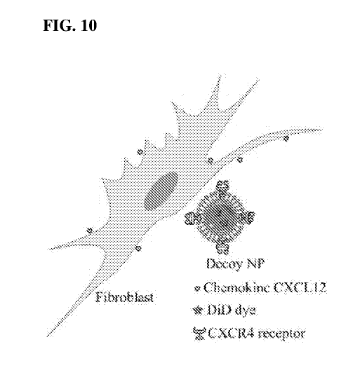

Also provided is the functionalization of degradable poly(lactic-co-glycolic acid) (PLGA) polymeric nanoparticles with a layer of cell membrane derived from CXCR4-overexpressed U87MG (U87-CXCR4) cells to form a core-shell nanostructure (Fang, R. et al., Nano Lett. 2014, 14, 2181-2188). Because of the specific expression of alpha smooth muscle actin (α-SMA) on CAFs, the membrane-coated NPs are labeled with antibodies against α-SMA to guide the NPs to attach to CAFs and act as a nanosponge that absorbs CXCL12 secreted by CAFs (schematic in FIG. 9 and FIG. 10). The parameters of the invention are set forth in additional detail below.

Described herein is the coating of synthetic polymeric nanoparticles (NPs) with plasma membranes derived from various cancer cells. The membrane-associated components (lipids, proteins, and carbohydrates) are retained in a native-like state within the cell membranes after isolation and translocation to the surface of NPs where all components present in the right-side-out orientation. This biomimetic strategy provides the advantage of replicating the complex surface of the cancer cell plasma membrane profile on NPs and consequently, this technology provides a robust means of using NPs as; e.g., decoys to misdirect cancer cell signaling or as cancer vaccines that activate immune responses to an individual's cancer along with a capacity to carry a range of therapeutic cargoes or imaging reporters for cell-specific delivery applications.

Described herein is the harnessing of cancer cell plasma membranes as biologically functional coatings for polymeric NPs. Cancer cell plasma membrane fractions possess a comprehensive array of antigens in native conformations, the complexity of which is unlikely to be duplicated by any synthetic chemistry or structural biology strategy. Being biomimetic means that these NPs possess natural attributes of the host's biology and as such have stealth-like properties, i.e., less immunogenicity than antigen presentation approaches prior to the invention described herein. Recent advances have demonstrated the feasibility of coating NPs with red blood cell membranes (RBCs) to mimic RBCs. However, described herein is technology that allows for coating of NPs with specific biologically functional cancer cell membranes. The utility of these biomimetic NPs is that they ae loaded with; e.g., therapeutic cargos for cell-specific targeted treatments or they can be used to assist in the activation of the immune response against a cancer or to disrupt/abrogate fatal cancer cell signaling/survival. Importantly, a distinct advantage is the use of a patient's cancer cells as the origin of the membranes for such strategies, which fully aligns with the concept of personalized medicine.

The biomimetic nanoparticle formulation technology consists of two components: (1) the plasma membrane fractions (MFs) of cancer cells isolated under a sequential process of hypotonic lysing, Potter-Elvehjem homogenization and Percoll® density gradient centrifugation, which allows for the isolation of pure plasma MFs as flexible bilayer vesicles with an average size of approximately 200 nm; and (2) polymeric NPs consisting of carboxy-terminated polylactic-co-glycolic acid (PLGA), an FDA-approved biodegradable polymer, which forms spherical negatively charged particles in the range of 40-60 nm through the processes of precipitation and evaporation. A far-red fluorescent dye, 1,1′-dioctadecyl-3,3,3′,3′-tetramethylindodicarbocyanine, 4-chlorobenzenesulfonate salt (DiD, ex/em: 644 nm/665 nm) is incorporated into the PLGA core for fluorescently tracking NPs. To generate biologically functional biomimetic nanoparticles, MFs and PLGA NPs are mixed and subjected to physical extrusion through a polycarbonate porous membrane. The extrusion process creates a uniform unilamellar MF coating with a thickness of 5 nm encapsulating the PLGA NPs. The mechanical force of the extrusion process guides the membrane-particle assembly while the electrostatic interaction between PLGA NPs and MFs enables the efficient and complete translocation of the fully functional plasma membrane with all of its associated components onto the polymeric NP surface in a “right-side-out” manner. This facile membrane coating approach is scalable and highly reproducible. As noted above, the utility of these biomimetic NPs is that they are loaded with; e.g., therapeutic cargos for cell-specific targeted treatments or they are used to assist in the activation of the immune response against a cancer or to disrupt/abrogate fatal cancer cell signaling/survival. Importantly, a distinct advantage is the use of a patient's cancer cells as the origin of the membranes for such strategies, which fully aligns with the concept of personalized medicine.

### Decoy Nanoparticles

Decoys are often employed to achieve distraction or misdirection. The development of decoy nanoparticles (NPs) that distract or misdirect cancer cells or cancer associated stromal cells results in a disruption of interactions between cancer cells and stromal cells. In some cases, the present invention provides for the development of biomimetic NPs consisting of FDA approved poly(lactic-co-glycolic acid) PLGA, covered with cancer cell membranes to act as decoys to misdirect or distract cancer cells, or cancer associated stromal cells. Once developed and characterized, the NPs are evaluated for their ability to attach to cancer cells, and activated fibroblasts in circulation, and at primary or distant tumor sites. In some cases, these NPs are decorated with an imaging reporter to characterize their biodistribution in vivo and ex vivo. Such NPs have not been previously developed for applications in cancer. The ultimate purpose was to determine if these NPs attract circulating cancer cells, circulating stromal cells, or disrupt the spontaneous or experimental metastatic cascade in triple negative breast cancer (TNBC). Stromal cells such as cancer associated fibroblasts (CAFs) mediate many of the aggressive characteristics of cancer (Horimoto Y, Polanska U M, Takahashi Y, Orimo A. Emerging roles of the tumor-associated stroma in promoting tumor metastasis. Cell Adh Migr. 2012; 6(3):193-202), but have an ever-replenishing supply that was largely left intact by therapeutic strategies prior to the invention described herein (Eyden B. The myofibroblast: phenotypic characterization as a prerequisite to understanding its functions in translational medicine. J Cell Mol Med. 2008; 12(1):22-37) (Eyden B, Banerjee S S, Shenjere P, Fisher C. The myofibroblast and its tumours. J Clin Pathol. 2009; 62(3):236-49). Therefore, even following surgery or chemotherapy, a few surviving cancer cells that ordinarily would not survive on their own continue to have a host of stromal cells to assist them in reestablishment, either at the primary site or at a distant site. Because of their important functional roles, destroying stromal cells that assist cancer cells is not a viable solution. Instead, as described herein, disrupting communications between cancer cells and stromal cells is a useful strategy. TNBCs are the most lethal breast cancers, and have limited treatment options. Since the CXCL12-CXCR4 axis has a wide spectrum of roles in facilitating breast cancer invasion and metastasis through breast cancer cell-CAF signaling, the role of high and low CXCR4 expressing cancer cell membrane coated NPs in disrupting cancer cell-CAF interactions is investigated as described in detail below. CAFs also play a major role in the formation of collagen 1 (Col1) fibers in tumors. Therefore, the functional effects of these NPs on Col1 fiber patterns in primary and metastatic tumors are also evaluated. In some embodiments, such NPs are loaded with a therapeutic cargo for targeting the premetastatic niche or eliminating circulating cancer cells, or they are used to assist in the activation of the immune response. These NPs are also labeled with magnetic resonance (MR) contrast agents or radiolabeled for detection using human MR or positron emission tomography (PET) scanners. These studies identify new, clinically translatable strategies to disrupt the metastatic cascade in breast cancer, and represent a new strategy in developing effective treatments to prevent metastatic breast cancer.

The present invention provides for the development and characterization of cancer cell membrane covered NPs that contain an optical imaging reporter. Cancer cell membranes from triple negative metastatic DU4475 and MDA-MB-231 human breast cancer cells are used in these studies. The present invention also provides for the evaluation of the interaction between the developed NPs and fibroblasts and cancer cells in terms of migration and binding in culture, and the determination of the effects on tumor growth, Col1 fiber formation, and metastasis.

As described in detail below, decoy NPs covered with cancer cell membranes mimic cancer cells and disrupt cancer cell-stromal cell interactions, reduce Col1 fiber formation in primary and metastatic tumors, and decrease the establishment of breast cancer metastasis.

Two triple (ER/PR/HER2) negative human breast cancer cell lines, DU4475 and MDA-MB-231 with high and low CXCR4 receptor expression (Nimmagadda S, Pullambhatla M, Stone K, Green G, Bhujwalla Z M, Pomper M G. Molecular imaging of CXCR4 receptor expression in human cancer xenografts with [64Cu]AMD3100 positron emission tomography. Cancer Res. 2010; 70(10):3935-4) are selected for these studies. In addition, MDA-MB-231 cells express the CD44 antigen (Krishnamachary B, Penet M F, Nimmagadda S, Mironchik Y, Raman V, Solaiyappan M, Semenza G L, Pomper M G, Bhujwalla Z M. Hypoxia regulates CD44 and its variant isoforms through HIF-1alpha in triple negative breast cancer. PLoS One. 2012; 7(8):e44078), a marker associated with stem-like breast cancer cells (Angeloni V, Tiberio P, Appierto V, Daidone M G. Implications of stemness-related signaling pathways in breast cancer response to therapy. Seminars in cancer biology. 2014), that provide additional validation of cell membrane integrity. Cancer cells from these two cell lines are used to form membrane vesicles to coat the NPs. The NPs also contain an imaging reporter.

Also provided are injectable NPs to disrupt the establishment of breast cancer metastasis in humans Biocompatibility is important, making the use of biomimetic NPs relevant. For example, the patient's own cancer cells are used to synthesize the NPs for personalized medicine. Following NP synthesis, characterization of toxicity, binding, stability and functional effects are performed in culture. These studies assist in identifying optimum doses for in vivo characterization that determine the effects of NPs on tumor growth, metastasis, Col1 fiber formation, and the presence of CAFs. The potential use of these NPs in identifying the premetastatic niche is also evaluated. Because of the critically important roles of stromal cells in several functions including the establishment of metastasis, strategies that disrupt the communications between cancer cells and stromal cells without destroying them provide solutions to prevent them from assisting cancer cells to survive, invade, and metastasize. In some cases, such NPs also carry targeting peptides and molecular reagents such as complementary deoxyribonucleic acid (cDNA) and small interfering ribonucleic acid (siRNA) to act as multiple signaling disruptors against a spectrum of stromal cells to disrupt cancer cell survival and the establishment of metastasis.

Also provided are decoy NPs that disrupt the interactions between cancer cells and stromal cells in an effort to define biomembrane coated NP based strategies to prevent or attenuate breast cancer metastasis.

Recent advances in polymeric NPs camouflaged in cellular membranes have paved the way for entirely new strategies in cancer (Hu C M, Fang R H, Copp J, Luk B T, Zhang L. A biomimetic nanosponge that absorbs pore-forming toxins. Nature nanotechnology. 2013; 8(5):336-40) (Fang R H, Hu C M, Chen K N, Luk B T, Carpenter C W, Gao W, Li S, Zhang D E, Lu W, Zhang L. Lipidinsertion enables targeting functionalization of erythrocyte membrane-cloaked nanoparticles. Nanoscale. 2013; 5(19):8884-8) (Hu C M, Fang R H, Luk B T, Zhang L. Polymeric nanotherapeutics: clinical development and advances in stealth functionalization strategies. Nanoscale. 2014; 6(1):65-75) (Luk B T, Jack Hu C M, Fang R H, Dehaini D, Carpenter C, Gao W, Zhang L. Interfacial interactions between natural RBC membranes and synthetic polymeric nanoparticles. Nanoscale. 2014; 6(5):2730-2737). These advances have demonstrated the feasibility of coating NPs in a ‘right-side’ out manner using red blood cell (RBC) membranes to mimic RBCs, and act as nanosponges for toxins. Here, for the first time, provided are NPs coated with cancer cell membranes, initially to act as nanosponges for CXCL12 in proof-of-principle studies, and to act as potential decoys. The major advantage is that the patient's cancer cells can be cultured and used for such strategies. If these NPs arrive at a premetastatic niche, they are also used to disrupt this niche, by carrying molecular targeting agents to prevent metastasis. The NPs also enhance immunotherapy strategies by presenting cell surface antigens. Described in detail below is the examination of two cell lines with different levels of CXCR4 expression to disrupt cancer cell-fibroblast interactions that play a major role in breast cancer metastasis.

### Plasma Membranes

Cell membranes contain a variety of biological molecules, notably lipids and proteins. Material is incorporated into the membrane, or deleted from it, by a variety of mechanisms: Fusion of intracellular vesicles with the membrane (exocytosis) not only excretes the contents of the vesicle, but also incorporates the vesicle membrane's components into the cell membrane. The membrane may form blebs around extracellular material that pinch off to become vesicles (endocytosis). If a membrane is continuous with a tubular structure made of membrane material, then material from the tube can be drawn into the membrane continuously. Although the concentration of membrane components in the aqueous phase is low (stable membrane components have low solubility in water), there is an exchange of molecules between the lipid and aqueous phases.

Examples of the major membrane phospholipids and glycolipids include phosphatidylcholine (PtdCho), phosphatidylethanolamine (PtdEtn), phosphatidylinositol (PtdIns), phosphatidylserine (PtdSer). The cell membrane consists of three classes of amphipathic lipids: phospholipids, glycolipids, and sterols. The amount of each depends upon the type of cell, but in the majority of cases phospholipids are the most abundant. In RBC studies, 30% of the plasma membrane is lipid. The fatty chains in phospholipids and glycolipids usually contain an even number of carbon atoms, typically between 16 and 20. The 16- and 18-carbon fatty acids are the most common. Fatty acids may be saturated or unsaturated, with the configuration of the double bonds nearly always “cis”. The length and the degree of unsaturation of fatty acid chains have a profound effect on membrane fluidity as unsaturated lipids create a kink, preventing the fatty acids from packing together as tightly, thus decreasing the melting temperature (increasing the fluidity) of the membrane. The ability of some organisms to regulate the fluidity of their cell membranes by altering lipid composition is called homeoviscous adaptation. The entire membrane is held together via non-covalent interaction of hydrophobic tails, however the structure is quite fluid and not fixed rigidly in place. Under physiological conditions, phospholipid molecules in the cell membrane are in the liquid crystalline state. This means the lipid molecules are free to diffuse and exhibit rapid lateral diffusion along the layer in which they are present. However, the exchange of phospholipid molecules between intracellular and extracellular leaflets of the bilayer is a very slow process.

Lipid rafts and caveolae are examples of cholesterol-enriched microdomains in the cell membrane. Also, a fraction of the lipid in direct contact with integral membrane proteins, which is tightly bound to the protein surface is called annular lipid shell; it behaves as a part of protein complex. In animal cells, cholesterol is normally found dispersed in varying degrees throughout cell membranes, in the irregular spaces between the hydrophobic tails of the membrane lipids, where it confers a stiffening and strengthening effect on the membrane. Lipid vesicles or liposomes are circular pockets that are enclosed by a lipid bilayer. These structures are used in laboratories to study the effects of chemicals in cells by delivering these chemicals directly to the cell, as well as getting more insight into cell membrane permeability. Lipid vesicles and liposomes are formed by first suspending a lipid in an aqueous solution then agitating the mixture through sonication, resulting in a vesicle. Membrane permeability is examined by measuring the rate of efflux from that of the inside of the vesicle to the ambient solution. Vesicles can be formed with molecules and ions inside the vesicle by forming the vesicle with the desired molecule or ion present in the solution. Proteins can also be embedded into the membrane through solubilizing the desired proteins in the presence of detergents and attaching them to the phospholipids in which the liposome is formed. These tools allow for the examination of various membrane protein functions.

Plasma membranes also contain carbohydrates, predominantly glycoproteins, but with some glycolipids (cerebrosides and gangliosides). For the most part, no glycosylation occurs on membranes within the cell; rather generally glycosylation occurs on the extracellular surface of the plasma membrane. The glycocalyx is an important feature in all cells, especially epithelia with microvilli. Recent data suggest the glycocalyx participates in cell adhesion, lymphocyte homing, and many other functions. The penultimate sugar is galactose and the terminal sugar is sialic acid, as the sugar backbone is modified in the golgi apparatus. Sialic acid carries a negative charge, providing an external barrier to charged particles.

The cell membrane has a large content of proteins, typically around 50% of membrane volume. These proteins are important for the cell because they are responsible for various biological activities. Approximately a third of the genes in yeast code specifically for cell membrane proteins, and this number is even higher in multicellular organisms. The cell membrane, being exposed to the outside environment, is an important site of cell-cell communication. As such, a large variety of protein receptors and identification proteins, such as antigens, are present on the surface of the membrane. Functions of membrane proteins can also include cell-cell contact, surface recognition, cytoskeleton contact, signaling, enzymatic activity, or transporting substances across the membrane. Most membrane proteins must be inserted in some way into the membrane. For this to occur, an N-terminus “signal sequence” of amino acids directs proteins to the endoplasmic reticulum, which inserts the proteins into a lipid bilayer. Once inserted, the proteins are then transported to their final destination in vesicles, where the vesicle fuses with the target membrane.

### Nanoparticles

Characterization of the nanoparticles described herein is necessary to establish understanding and control of nanoparticle synthesis and applications. Characterization is done by using a variety of different techniques, mainly drawn from materials science. Common techniques include electron microscopy (transmission electron microscopy (TEM), scanning electron microscopy (SEM)), atomic force microscopy (AFM), dynamic light scattering (DLS), x-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, Rutherford backscattering spectrometry (RBS), dual polarisation interferometry and nuclear magnetic resonance (NMR). The technology for nanoparticle tracking analysis (NTA) allows direct tracking of the Brownian motion, which allows the sizing of individual nanoparticles in solution. The majority of these nanoparticle characterization techniques are light-based, but a non-optical nanoparticle characterization technique called Tunable Resistive Pulse Sensing (TRPS) has been developed that enables the simultaneous measurement of size, concentration and surface charge for a wide variety of nanoparticles. This technique, which applies the Coulter Principle, allows for particle-by-particle quantification of these three nanoparticle characteristics with high resolution.

The surface coating of nanoparticles is crucial to determining their properties. In particular, the surface coating can regulate stability, solubility, and targeting. A coating that is multivalent or polymeric confers high stability. Functionalized nanomaterial-based catalysts can be used for catalysis of many known organic reactions.

For biological applications, the surface coating should be polar to give high aqueous solubility and prevent nanoparticle aggregation. In serum or on the cell surface, highly charged coatings promote non-specific binding, whereas polyethylene glycol linked to terminal hydroxyl or methoxy groups repel non-specific interactions. Nanoparticles can be linked to biological molecules that can act as address tags, to direct the nanoparticles to specific sites within the body, specific organelles within the cell, or to follow specifically the movement of individual protein or RNA molecules in living cells. Common address tags are monoclonal antibodies, aptamers, streptavidin or peptides. These targeting agents should ideally be covalently linked to the nanoparticle and should be present in a controlled number per nanoparticle. Multivalent nanoparticles, bearing multiple targeting groups, can cluster receptors, which can activate cellular signaling pathways, and give stronger anchoring. Monovalent nanoparticles, bearing a single binding site, avoid clustering and so are preferable for tracking the behavior of individual proteins.

### Methods of Treating Diseases

Provided herein are methods of treating diseases, disorders or conditions associated with cancer cell-stromal cell networks. Compositions described herein are used to stimulate and activate the immune response to cancer cells by exposing the immune system to nanoparticles coated with plasma membranes derived from cancer cells. Furthermore, pre-exposing the immune system to nanoparticles coated in plasma membranes derived from cancer cells acts as a vaccination against those types of cancer.

Compositions of the present invention include nanoparticles as delivery agents. Compositions are used to deliver: therapies, drugs, pharmaceutical compositions, isotopes, and any combination thereof.

Compositions of the present invention are administered to subjects in a variety of routes including but not limited to: oral administration, intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, or subcutaneous administration.

Compositions of the present invention are administered to subjects in a variety of forms including but not limited to: pills, capsules, tablets, granules, powders, salts, crystals, liquids, serums, syrups, solutions, emulsions, suspensions, gels, creams, pastes, films, patches, and vapors.

### Cancer

Cancers are a large family of diseases that involve abnormal cell growth with the potential to invade or spread to other parts of the body. They form a subset of neoplasms. A neoplasm or tumor is a group of cells that have undergone unregulated growth, and will often form a mass or lump, but may be distributed diffusely. Six characteristics of cancer have been proposed: self-sufficiency in growth signaling; insensitivity to anti-growth signals; evasion of apoptosis; enabling of a limitless replicative potential; induction and sustainment of angiogenesis; and activation of metastasis invasion of tissue. The progression from normal cells to cells that can form a discernible mass to outright cancer involves multiple steps known as malignant progression.

For example, the methods described herein are useful in treating various types of malignancies and/or tumors, e.g., non-Hodgkin's lymphoma (NHL), acute lymphocytic leukemia (ALL), acute myeloid leukemia (AML), chronic lymphocytic leukemia (CLL), chronic myelogenous leukemia (CML), multiple myeloma (MM), breast cancer, ovarian cancer, head and neck cancer, bladder cancer, melanoma, colorectal cancer, pancreatic cancer, lung cancer, leiomyoma, leiomyosarcoma, glioma, and glioblastoma. Solid tumors include, e.g., breast tumors, ovarian tumors, lung tumors, pancreatic tumors, prostate tumors, melanoma tumors, colorectal tumors, lung tumors, head and neck tumors, bladder tumors, esophageal tumors, liver tumors, and kidney tumors.

### Stromal Cells

Stromal cells are connective tissue cells of any organ, for example in the uterine mucosa (endometrium), prostate, bone marrow, and the ovary. They are cells that support the function of the parenchymal cells of that organ. Fibroblasts and pericytes are among the most common types of stromal cells. The interaction between stromal cells and tumor cells is known to play a major role in cancer growth and progression. In addition, by regulating locally cytokine networks (e.g. M-CSF, LIF), bone marrow stromal cells have been described to be involved in human hematopoiesis and inflammatory processes. Stromal cells (in the dermis layer) adjacent to the epidermis (the very top layer of the skin) release growth factors that promote cell division. This keeps the epidermis regenerating from the bottom while the top layer of cells on the epidermis are constantly being “sloughed” off of the body. Certain types of skin cancers (basal cell carcinomas) cannot spread throughout the body because the cancer cells require nearby stromal cells to continue their division. The loss of these stromal growth factors when the cancer moves throughout the body prevents the cancer from invading other organs. Stroma is made up of the non-malignant host cells. Stroma provides an extracellular matrix on which tumors can grow.

### Immunotherapy

In some embodiments, the present invention provides for methods of treating cancer based on immunotherapy. Immunotherapy is the treatment of disease by inducing, enhancing, or suppressing an immune response. Immunotherapies designed to elicit or amplify an immune response are classified as activation immunotherapies, while immunotherapies that reduce or suppress are classified as suppression immunotherapies. Cancer immunotherapy (immuno-oncology) is the use of the immune system to treat cancer. Immunotherapies fall into three main groups: cellular, antibody and cytokine. They exploit the fact that cancer cells often have subtly different molecules on their surface that can be detected by the immune system. These molecules, known as cancer antigens, are most commonly proteins, but also include molecules such as carbohydrates. Immunotherapy is used to provoke the immune system into attacking the tumor cells by using these antigens as targets.

Antibody therapies are the most successful immunotherapy, treating a wide range of cancers. Antibodies are proteins produced by the immune system that bind to a target antigen on the cell surface. In normal physiology, the immune system uses antibodies to fight pathogens. Each antibody is specific to one or a few proteins. Those that bind to cancer antigens are used to treat cancer. Cell surface receptors, e.g., CD20, CD274, and CD279, are common targets for antibody therapies. Once bound to a cancer antigen, antibodies can induce antibody-dependent cell-mediated cytotoxicity, activate the complement system, or prevent a receptor from interacting with its ligand, all of which can lead to cell death. Multiple antibodies are approved to treat cancer, including Alemtuzumab, Ipilimumab, Nivolumab, Ofatumumab, and Rituximab.

Cellular therapies, also known as cancer vaccines, usually involve the removal of immune cells from the blood or from a tumor. Immune cells specific for the tumor are activated, cultured and returned to the patient where the immune cells attack the cancer. Cell types that can be used in this way are natural killer cells, lymphokine-activated killer cells, cytotoxic T cells and dendritic cells.

Interleukin-2 and interferon-α are examples of cytokines, proteins that regulate and coordinate the behavior of the immune system. They have the ability to enhance anti-tumor activity and thus can be used as cancer treatments. Interferon-α is used in the treatment of hairy-cell leukemia, AIDS-related Kaposi's sarcoma, follicular lymphoma, chronic myeloid leukemia and malignant melanoma. Interleukin-2 is used in the treatment of malignant melanoma and renal cell carcinoma.

### Disease Detection

Also described herein are methods of detecting cancer in a subject. For example, described herein are compositions comprising plasma membrane derived vesicles fused to nanoparticles further comprising a detectable label. Such labels include, but are not limited to: radioisotopes, isotopes, contrast agents, metals, and fluorescent dyes. The compositions of the present invention are used in imaging modalities including but not limited to: fluorescent imaging, fluorescent tomography, computed tomography, magnetic resonance imaging, positron emission tomography, x-ray tomography, ultrasound, and any combinations thereof.

This invention is further illustrated by the following examples which should not be construed as limiting. The contents of all references, patents, and published patent applications cited throughout this application, as well as the figures, are incorporated herein by reference.

### EXAMPLES

### Example 1: Role of CAFs and CXCL12 in Facilitating Breast Cancer Metastasis

Metastasis continues to be a major cause of mortality from breast cancer. The process of metastasis is multidirectional and cancer cells seed distant sites and the primary tumor (Comen E A. Tracking the seed and tending the soil: evolving concepts in metastatic breast cancer. Discovery medicine. 2012; 14(75):97-104) (Oskarsson T, Batlle E, Massague J. Metastatic stem cells: sources, niches, and vital pathways. Cell stem cell. 2014; 14(3):306-21) (Goubran H A, Kotb R R, Stakiw J, Emara M E, Burnouf T. Regulation of tumor growth and metastasis: the role of tumor microenvironment. Cancer growth and metastasis. 2014; 7:9-18). Several steps in this multidirectional process require the assistance of stromal cells and occur through the blood stream providing opportunities for disruption and misdirection by opportunistic circulating NPs and by NPs that arrest in existing primary or distant tumor sites or pre-metastatic niches that support the survival of disseminated cancer cells (Oskarsson T, Batlle E, Massague J. Metastatic stem cells: sources, niches, and vital pathways. Cell stem cell. 2014; 14(3):306-21). The stromal cells that play a major role include activated fibroblasts and tumor associated macrophages (Hanahan D, Coussens L M. Accessories to the crime: functions of cells recruited to the tumor microenvironment. Cancer cell. 2012; 21(3):309-22) (Ohlund D, Elyada E, Tuveson D. Fibroblast heterogeneity in the cancer wound. The Journal of experimental medicine. 2014; 211(8):1503-23) (Heusinkveld M, van der Burg S H. Identification and manipulation of tumor associated macrophages in human cancers. Journal of translational medicine. 2011; 9:216). Resident fibroblasts in connective tissues adjacent to cancer cells, or local and bone marrow derived mesenchymal stem cells are recruited and induced to form activated fibroblasts or myoblasts (Hanahan D, Coussens L M. Accessories to the crime: functions of cells recruited to the tumor microenvironment. Cancer cell. 2012; 21(3):309-22) (Ohlund D, Elyada E, Tuveson D. Fibroblast heterogeneity in the cancer wound. The Journal of experimental medicine. 2014; 211(8):1503-23). Cancer cells also attract myeloid cells that are differentiated into tumor promoting macrophages (Heusinkveld M, van der Burg S H. Identification and manipulation of tumor associated macrophages in human cancers. Journal of translational medicine. 2011; 9:216).

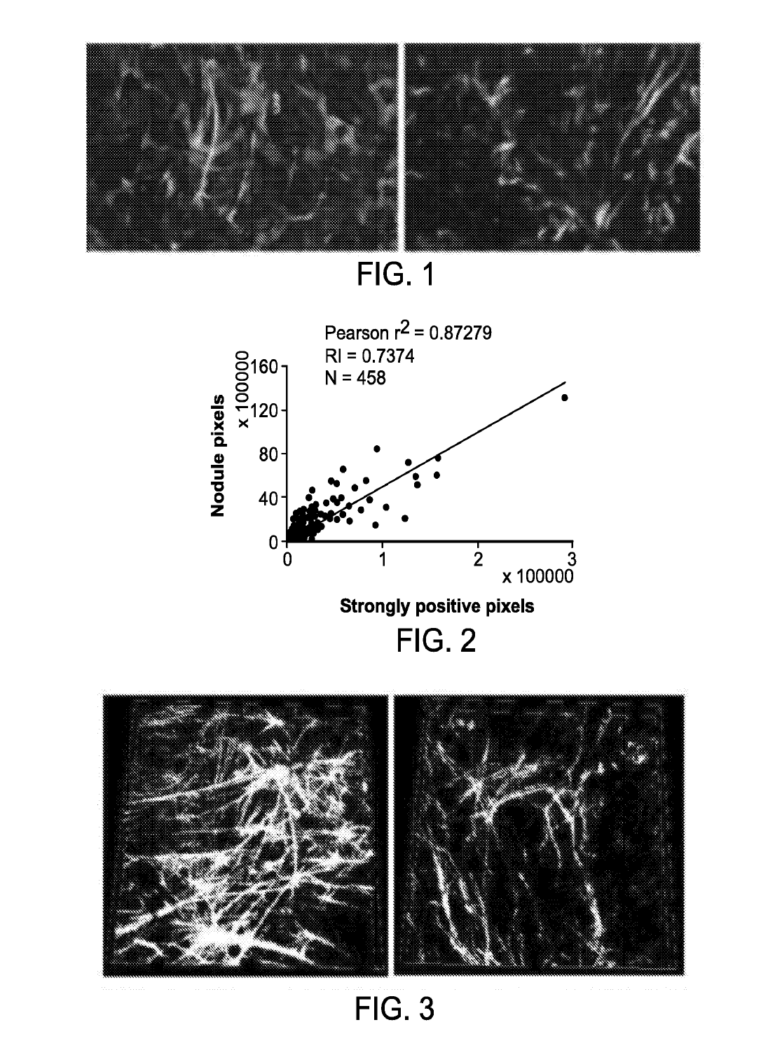

The activated fibroblast or myoblast is a versatile cell that plays an active role in wound healing and is present in fibroproliferative conditions and in cancers (Eyden B. The myofibroblast: phenotypic characterization as a prerequisite to understanding its functions in translational medicine. J Cell Mol Med. 2008; 12(1):22-37) (Eyden B, Banerjee S S, Shenjere P, Fisher C. The myofibroblast and its tumours. J Clin Pathol. 2009; 62(3):236-49). Fibroblasts are being increasingly investigated as a therapeutic target in cancer (Togo S, Polanska U M, Horimoto Y, Orimo A. Carcinoma-associated fibroblasts are a promising therapeutic target. Cancers (Basel). 2013; 5(1):149-69). CAFs are usually activated and are positive for α-SMA (smooth muscle actin) (Cirri P, Chiarugi P. Cancer associated fibroblasts: the dark side of the coin. Am J Cancer Res. 2011; 1(4):482-97). There are several sources of CAFs. These include resident normal fibroblasts, endothelial cells, pericytes, smooth muscle cells, preadipocytes and bone marrow derived progenitors, such as fibrocytes and mesenchymal stem cells (Horimoto Y, Polanska U M, Takahashi Y, Orimo A. Emerging roles of the tumor-associated stroma in promoting tumor metastasis. Cell Adh Migr. 2012; 6(3):193-202) (Eyden B. The myofibroblast: phenotypic characterization as a prerequisite to understanding its functions in translational medicine. J Cell Mol Med. 2008; 12(1):22-37) (Polanska U M, Orimo A. Carcinoma-associated fibroblasts: non-neoplastic tumour-promoting mesenchymal cells. J Cell Physiol. 2013; 228(8):1651-7). Recent studies have identified the bone marrow as a major source of CAFs (McDonald L T, LaRue A C. Hematopoietic stem cell derived carcinoma-associated fibroblasts: a novel origin. Int J Clin Exp Pathol. 2012; 5(9):863-73). Poorly metastatic primary tumors are less able to attract circulating fibroblasts as shown in FIG. 1.

A strong association was identified between CAFs and the size of metastatic nodules in the lungs as shown in FIG. 2. These data suggest that CAFs and Col1 fibers are important for metastatic growth. CAFs play an active role in breast cancer metastasis through the expression of CXCL12 (also called SDF-1) (Mao Y, Keller E T, Garfield D H, Shen K, Wang J. Stromal cells in tumor microenvironment and breast cancer. Cancer Metastasis Rev. 2013; 32(1-2):303-15) (Orimo A, Gupta P B, Sgroi D C, Arenzana-Seisdedos F, Delaunay T, Naeem R, Carey V J, Richardson A L, Weinberg R A. Stromal fibroblasts present in invasive human breast carcinomas promote tumor growth and angiogenesis through elevated SDF-1/CXCL12 secretion. Cell. 2005; 121(3):335-48). CXCL12 is a homeostatic chemokine that signals through CXCR4, which is part of the family of chemokine receptors that induce directional migration of cells toward a gradient of a chemotactic cytokine through autocrine and paracrine signaling (Burger J A, Kipps T J. CXCR4: a key receptor in the crosstalk between tumor cells and their microenvironment. Blood. 2006; 107(5):1761-7). CXCL12 is a highly conserved chemokine that has 99% homology between mouse and man (Burger J A, Kipps T J. CXCR4: a key receptor in the crosstalk between tumor cells and their microenvironment. Blood. 2006; 107(5):1761-7). Breast CAFs secrete high levels of CXCL12, thereby stimulating both the mobilization of endothelial progenitor cells from the bone marrow and promoting growth by binding to CXCR4 expressed on the surface of breast carcinoma cells (Orimo A, Gupta P B, Sgroi D C, Arenzana-Seisdedos F, Delaunay T, Naeem R, Carey V J, Richardson A L, Weinberg R A. Stromal fibroblasts present in invasive human breast carcinomas promote tumor growth and angiogenesis through elevated SDF-1/CXCL12 secretion. Cell. 2005; 121(3):335-48). The role of CXCR4 signaling and CXCL12 chemotaxis in breast cancer cell migration and metastasis is well established (Smith M C, Luker K E, Garbow J R, Prior J L, Jackson E, Piwnica-Worms D, Luker G D. CXCR4 regulates growth of both primary and metastatic breast cancer. Cancer Res. 2004; 64(23):8604-12) (Muller A, Homey B, Soto H, Ge N, Catron D, Buchanan M E, McClanahan T, Murphy E, Yuan W, Wagner S N, Barrera J L, Mohar A, Verastegui E, Zlotnik A. Involvement of chemokine receptors in breast cancer metastasis. Nature. 2001; 410(6824):50-6). Studies have also identified the role of CAFs in co-metastasizing with carcinoma cells, indicating that CAFs are directly involved in metastasis (Duda D G, Duyverman A M, Kohno M, Snuderl M, Steller E J, Fukumura D, Jain R K. Malignant cells facilitate lung metastasis by bringing their own soil. Proc Natl Acad Sci USA. 2010; 107(50):21677-82), and travel with cancer cells through the circulation on their metastatic journey. This data highlights the importance of CAFs and targeting the CAF-cancer cell interaction in preventing or reducing metastasis from breast cancer.

### Example 2: Role of CAFs in Col1 Fiber Formation in Breast Cancer

CAFs are a major source of Col1 fibers in the tumor stroma and contribute to the reactive desmoplastic tumor stroma, and the high density and stiffness of the tumor extracellular matrix (ECM) (Byun J S, Gardner K. Wounds that will not heal: pervasive cellular reprogramming in cancer. Am J Pathol. 2013; 182(4):1055-64). Like CAFs, Col1 fibers in breast cancer are actively investigated for their role in promoting metastasis (Lyons T R, O'Brien J, Borges V F, Conklin M W, Keely P J, Eliceiri K W, Marusyk A, Tan A C, Schedin P. Postpartum mammary gland involution drives progression of ductal carcinoma in situ through collagen and COX-2. Nat Med. 2011; 17(9):1109-15) (Conklin M W, Eickhoff J C, Riching K M, Pehlke C A, Eliceiri K W, Provenzano P P, Friedl A, Keely P J. Aligned collagen is a prognostic signature for survival in human breast carcinoma. Am J Pathol. 2011; 178(3):1221-32) (Provenzano P P, Eliceiri K W, Campbell J M, Inman D R, White J G, Keely P J. Collagen reorganization at the tumor-stromal interface facilitates local invasion. BMC Med. 2006; 4(1):38).

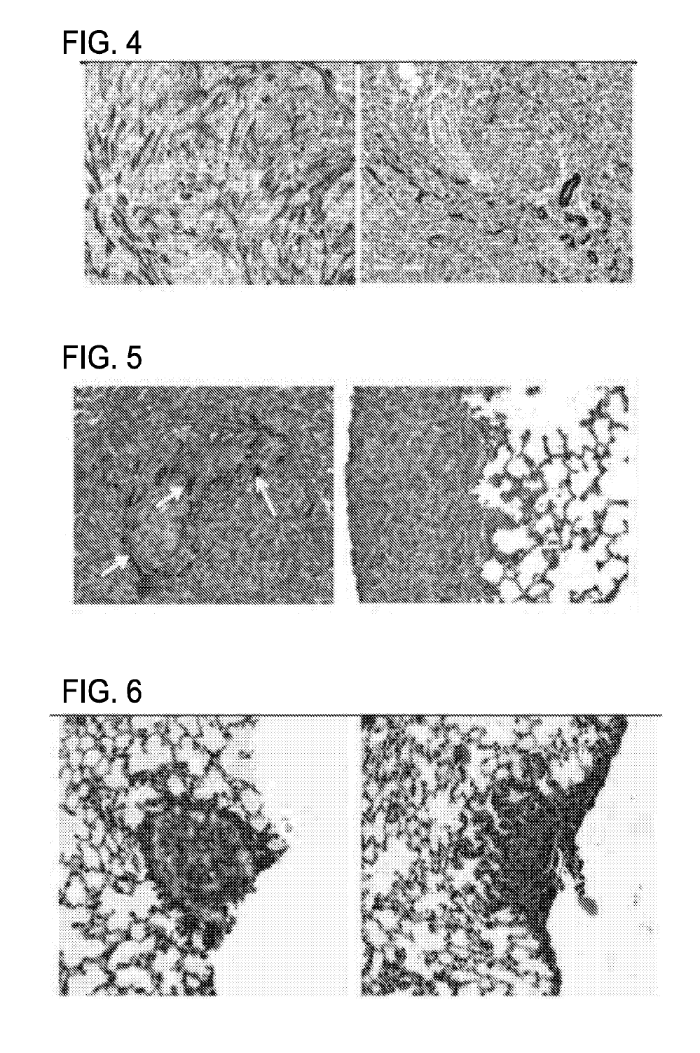

It was recently observed that significantly more Col1 fiber density in primary human breast cancers that were lymph node positive compared to those that were lymph node negative (Kakkad S M, Solaiyappan M, Argani P, Sukumar S, Jacobs L K, Leibfritz D, Bhujwalla Z M, Glunde K. Collagen I fiber density increases in lymph node positive breast cancers: pilot study. J Biomed Opt. 2012; 17(11):116017). Col1 secreted by CAFs also contributes to decreasing chemotherapeutic drug uptake in tumors and plays a significant role in regulating tumor sensitivity to a variety of chemotherapies (Loeffler M, Kruger J A, Niethammer A G, Reisfeld R A. Targeting tumor-associated fibroblasts improves cancer chemotherapy by increasing intratumoral drug uptake. J Clin Invest. 2006; 116(7):1955-62). Comparisons have been made concerning Col1 fibers in orthotopically and subcutaneously implanted tumors derived from identical prostate cancer cells that are highly metastatic (orthotopic) or poorly metastatic (subcutaneous) based on the site of inoculation. Furthermore, Col1 fibers in highly metastatic MDA-MB-231 tumors and poorly metastatic clones with COX-2 downregulated were also compared. Irrespective of the molecular pathway, decreased metastatic ability was closely associated with significantly reduced Col1 fiber density (FIG. 3) and a reduction of CAFs (FIG. 4). These differences in CAFs and Col1 fiber density were evident in metastatic lung nodules as well (FIGS. 5 and 6). These data indicate that CAFs play an important role in the establishment of metastasis and in the formation of Col1 fibers in primary and metastatic sites. These data also indicate the likelihood of detecting functional effects of reducing cancer cell-CAF interactions through changes in Col1 fibers.

Stromal cells, such as CAFs, mediate many of the aggressive characteristics of cancer (Dumont N, Liu B, Defilippis R A, Chang H, Rabban J T, Karnezis A N, Tjoe J A, Marx J, Parvin B, Tlsty T D. Breast fibroblasts modulate early dissemination, tumorigenesis, and metastasis through alteration of extracellular matrix characteristics. Neoplasia. 2013; 15(3):249-62) and have replenishing sources that are largely left intact by current therapeutic strategies. Because of the critically important roles of stromal cells in several functions, strategies that disrupt the communications between cancer cells and stromal cells without destroying them would provide solutions to prevent them from assisting cancer cells to survive, invade and metastasize. As described herein, multi-functional NPs that are decorated with different targeting moieties and that carry multiple signaling disruptors fill an important niche in disrupting cancer survival strategies.

Described herein are nanoparticles that focus on CAFs and the CXCR4-CXCL12 axis, as through this axis fibroblasts have a wide spectrum of interactions with cancer cells, other stromal cells, and immune cells in mediating breast cancer growth and metastasis (Liao D, Luo Y, Markowitz D, Xiang R, Reisfeld R A. Cancer associated fibroblasts promote tumor growth and metastasis by modulating the tumor immune microenvironment in a 4T1 murine breast cancer model. PLoS One. 2009; 4(11):e7965) (Silzle T, Kreutz M, Dobler Mass., Brockhoff G, Knuechel R, Kunz-Schughart L A. Tumor-associated fibroblasts recruit blood monocytes into tumor tissue. Eur J Immunol. 2003; 33(5):1311-20). The NPs are coated with cancer cell membranes that have high or low CXCR4 expression. To further facilitate the binding of the NPs to CAFs, there is the option of attaching fibroblast activation protein-α (FAP-α) antibody to the NPs, since FAP-α is selectively produced by CAFs and has been used to image CAFs in vivo (Li J, Chen K, Liu H, Cheng K, Yang M, Zhang J, Cheng J D, Zhang Y, Cheng Z. Activatable near-infrared fluorescent probe for in vivo imaging of fibroblast activation protein-alpha. Bioconjug Chem. 2012; 23(8):1704-11).

### Example 3: Synthesis of Decoy NPs

Degradable poly(lactic-co-glycolic acid) PLGA polymeric NPs has been functionalized with a layer of cell membrane derived from CXCR4-overexpressing U87MG (U87-CXCR4) cells to form a core-shell nanostructure. The plasma membrane fractions were isolated under a sequential process of homogenization and Percoll® density gradient centrifugation. PLGA polymeric NPs were produced by nanoprecipitation. The NPs and all the intermediate materials were characterized by transmission electron microscopy (TEM) and dynamic laser scattering (DLS) to reveal their size and morphological information, and probed by western blot with antibodies against plasma membrane markers (pan-cadherin and Na+/K+-ATPase), CXCR4, and cytosol marker (GAPDH). The plasma membrane fractions were stained with PE (phycoerythin)-conjugated anti-human CXCR4 antibody and checked with fluorescence microscopy.

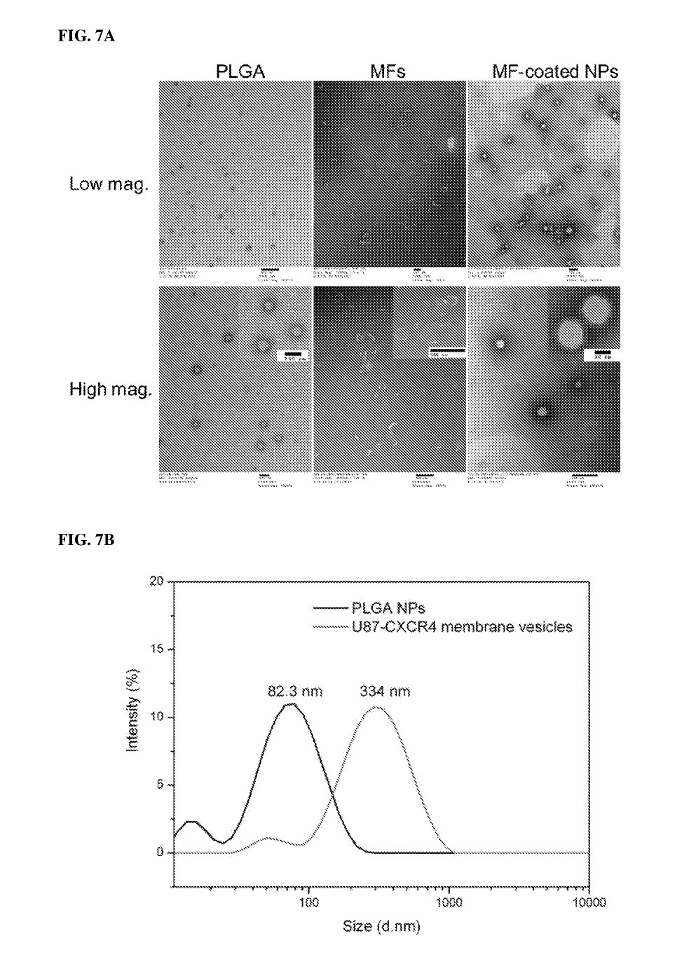

FIG. 7A displays the representative TEM images of PLGA NPs, U87-CXCR4 cell derived membrane vesicles (CDMVs) and U87-CXCR4 membrane-coated decoy NPs, with diameters of 50 nm, 150 nm, and 70 nm, respectively. These figures show that the U87-CXCR4 plasma membrane coated on the PLGA NPs has a thickness of around 10 nm. The DLS size intensity curves shown in FIG. 7B represent the size intensity distribution profile of PLGA NPs and U87-CXCR4 CDMVs. The Z-average diameters and polydispersity index of PLGA NPs and U87-CXCR4 CDMVs were measured to be 54.4 nm and 242 nm, and 0.28 and 0.271, respectively.

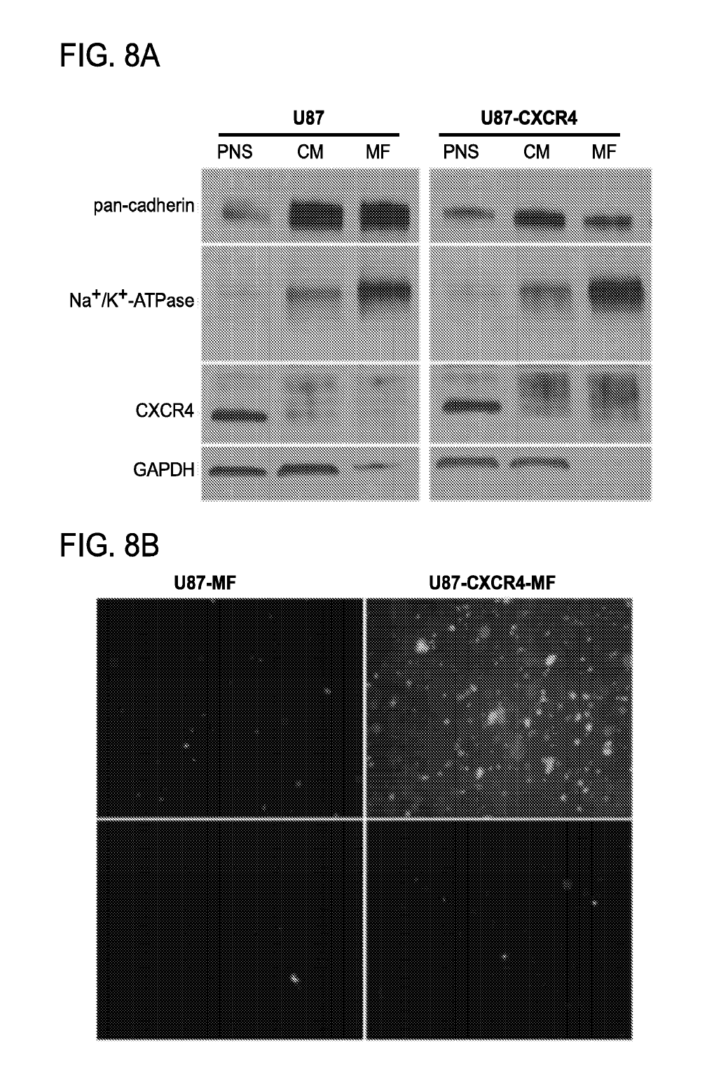

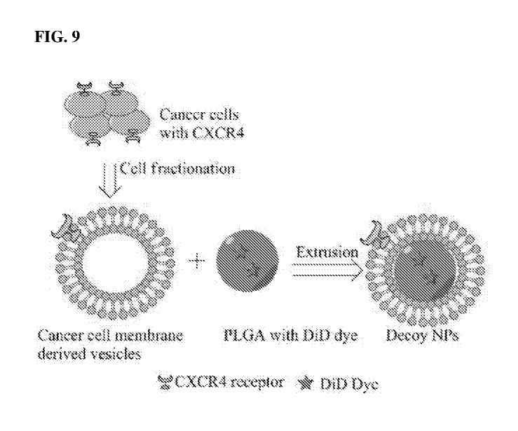

The protein content of the U87-CXCR4 cell fractions, including post nuclear supernatant (PNS), crude membrane (CM) and plasma membrane fraction (MF) were analyzed by western blots, and the results are presented in FIG. 8A with U87MG cell compartments for comparison. The CXCR4 receptors are present to a much lesser extent in U87MG cell MF compared to U87-CXCR4 MF. Confirming the vesicle formation, there was a significant enrichment of pan-cadherin and Na+/K+-ATPase, both plasma membrane markers, in U87MG and U87-CXCR4 MF when compared with the corresponding PNS and CM components. The negligible presence of glyceraldehyde 3-phosphate dehydrogenase (GAPDH) in MF indicates the high purity and low contamination from cytosolic components. U87-CXCR4 cell MF stained with PE-conjugated anti-human CXCR4 antibody shows much more intense fluorescence than U87 MF (FIG. 8B), confirming the successful retention of over-expressed CXCR4 on the surface of U87-CXCR4 MF. These data demonstrate the feasibility of preparing PLGA NPs, U87-CXCR4 CDMVs and U87-CXCR4 cancer cell membrane-coated NPs with appropriate size and surface receptor properties.

### Example 4: Nanoparticle μLatform Development and Characterization

A schematic outlining the preparation of membrane-coated PLGA NPs is shown in FIG. 9. To synthesize decoy NPs with cancer cell membrane coating, the plasma membrane fraction of cancer cells is isolated under a sequential process of homogenization and gradient-density centrifugation. Membrane fractions are extruded through 100-nm polycarbonate porous membranes to obtain membrane-derived vesicles. These membrane coated NPs act as nanosponges for CXCL12 (FIG. 10).

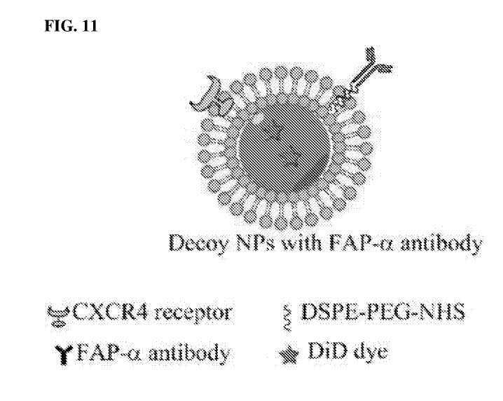

To specifically target NPs to CAFs, FAP-α antibody is attached to the vesicles (FIG. 11). FAP-α is a cell surface glycoprotein and a member of the serine protease family that has been found to be selectively produced by CAFs and has been used to image CAFs in vivo (Li J, Chen K, Liu H, Cheng K, Yang M, Zhang J, Cheng J D, Zhang Y, Cheng Z. Activatable near-infrared fluorescent probe for in vivo imaging of fibroblast activation protein-alpha. Bioconjug Chem. 2012; 23(8):1704-11).

DSPE-PEG-NHS (DSPE: 1,2-distearoyl-sn-glycero-3-phosphoethanolamine) is first reacted with FAP-α antibody at the molar ratio of 2:1 to maintain roughly one DSPE tail per antibody, and the resultant lipid modified FAP-α antibody is fused into the membrane derived vesicles through nonpolar hydrophobic interactions. For the preparation of PLGA polymeric cores, carboxy-terminated 50:50 poly(DL-lactide-co-glycolide) is first dissolved in acetone at a 1 mg/mL concentration. One milliliter (1 mL) of the acetone solution is added to 3 mL of water, and the mixture solution is subjected to rigorous stirring in open air for 2 h to allow the evaporation of acetone and the formation of the PLGA NPs through nanoprecipitation. The resulting NP solution is finally filtered with a 10 K molecular weight cutoff (MWCO) Amicon Centrifugal Filters. For fluorescently tracking the decoy NPs, a far-red fluorescent dye, 1,1′-dioctadecyl-3,3,3′,3′-tetramethylindodicarbocyanine, 4-chlorobenzenesulfonate salt (DiD, ex/em: 644 nm/665 nm) dye is added into the acetone solution for incorporation into the PLGA cores. To fuse the membrane-derived vesicles with the PLGA NPs, 1 mg of PLGA nanoparticles are mixed with membrane-derived vesicles and physically extruded several times through a 100-nm polycarbonate porous membrane. The PLGA NPs cross the lipid bilayers under the mechanical force to direct the membrane particle assembly.

The decoy NPs together with their control groups and all the intermediate materials, such as bare PLGA NPs and membrane-derived vesicles are characterized by TEM and DLS to reveal their size, morphology, and zeta potential information. The membrane-derived vesicles and decoy NPs are probed by western blot analysis with antibodies against an array of protein markers, pan-cadherin or Na+/K+-ATPase (plasma membrane), CXCR4, HSP-90 (cytosolic protein), and calreticulin (endoplasmic reticulum). Additionally, CD44 antibodies are used to evaluate membrane-derived vesicles obtained from MDA-MB-231 cells. In order to optimize the membrane coating, the decoy NPs are prepared with different membrane-to-PLGA weight ratios, and evaluated on the stability in PBS and the membrane coverage by measuring the hydrodynamic diameters with DLS. The ability of the NPs to act as nanosponges for CXCL12 are first tested in medium containing CXCL12 (˜500 mg/ml plasma level concentration) to determine if adding NPs reduces free CXCL12 concentration in medium.

### Example 5: Experimental Design and Methods

The following materials and methods were utilized for Examples 1-4.

### Cell Lines and Tumor Implantation

Two TNBC cell lines, DU4475 and MDA-MB-231 cell lines, are studied in culture and in vivo following orthotopic implantation (2×10⁶ cells in Hanks balanced salt solution) in the right upper thoracic mammary fat pad of female SCID (severe combined immunodeficient) mice. Since the in vivo studies are performed in mice, mouse and human fibroblasts are used for additional NP characterization. NIH 3T3 mouse fibroblasts that express tdTomato red fluorescent protein and immortalized human mammary fibroblasts that have a nuclear mCherry red fluorescent protein and a green fluorescent protein marker for their expression of telomerase are used.

### Studies in Culture

Once the NPs are synthesized, they are characterized in terms of: binding to activated NIH 3T3 fibroblasts and human mammary fibroblasts, toxicity to fibroblasts and cancer cells, stability in serum, and ability to disrupt cancer cell migration towards fibroblasts or cancer cells. Since CXCL12 and CXCR4 have 99% homology between mouse and human, these studies allow the evaluation of the feasibility of these strategies with human mammary fibroblasts. Binding of NPs to activated fibroblasts are determined using electron microscopy and optical imaging (from the ratio of optical marker in the NP to the optical marker in the fibroblast), by adding known concentrations of NPs to known numbers of activated fibroblasts, and observing the binding of the NPs following washing over a period of 5 days. Fibroblasts are activated in culture following exposure to TGF (tumor growth factor)-β (2 ng/ml for 72 h) (Chen H, Yang W W, Wen Q T, Xu L, Chen M. TGF-beta induces fibroblast activation protein expression; fibroblast activation protein expression increases the proliferation, adhesion, and migration of HO-8910PM [corrected]. Exp Mol Pathol. 2009; 87(3):189-94). These binding studies identify the duration of binding of the NPs to fibroblasts in culture. Fibroblast and cancer cell toxicity studies with the NPs, in the two TNBC cell lines are used in vivo, are performed using TUNEL (for apoptosis) and MTT (for viability) assays. Stability in serum is characterized by DLS of NPs after being maintained in serum for 24-72 h.

By adding known concentrations of NPs to known numbers of activated fibroblasts, fluorescence signal of the DiD dye on the surface of the fibroblast after washing is observed and recorded. Besides the membrane binding of decoy NPs to fibroblast, a certain degree of endocytosis of the NPs in fibroblasts and cancer cells is anticipated depending upon the duration of incubation. The fluorescence signal ratio between membrane bound and intracellular NPs is determined by confocal fluorescence imaging and μlotted as a function of incubation duration to reveal the best timeframe for maximizing binding. The intracellular localization of the decoy NPs is determined by co-staining with specific organelle fluorescence trackers, such as LysoTracker Green. To investigate the integrity of the membrane-coated NPs upon binding and internalization, the PLGA core is incorporated with DiD dye and the membrane shell is fluorescently labeled with NHS-fluorescein, respectively. Upon uptake, the overlapping degree of two fluorescence signals indicate the integrity of the NPs in cells. These data provide the framework for the dosing schedule and time course of the in vivo studies.

The ability of cancer cells to migrate to fibroblasts, or fibroblasts to migrate to cancer cells, or cancer cells to migrate to cancer cells are determined using a co-culture wound assay (Chen H, Yang W W, Wen Q T, Xu L, Chen M. TGF-beta induces fibroblast activation protein expression; fibroblast activation protein expression increases the proliferation, adhesion, and migration of HO-8910PM [corrected]. Exp Mol Pathol. 2009; 87(3):189-94) and confocal microscopy, flow cytometry, or a noninvasive imaging based assay that allows the dynamic visualization of co-cultured cells (Gimi B, Mori N, Ackerstaff E, Frost E E, Bulte J W, Bhujwalla Z M. Noninvasive MRI of endothelial cell response to human breast cancer cells. Neoplasia. 2006; 8(3):207-13) in the presence or absence of the NPs.

### Groups

All cell culture and in vivo experiments have the following groups for DU4475 and MDA-MB-231 cells: (i) saline only, (ii) cancer cell membrane coated NPs without FAP-α antibody, (iii) cancer cell membrane NPs with FAP-α antibody and (iv) FAP-α antibody alone, for comparison. In vivo, DU4475 cancer cell membrane coated NPs are used for DU4475 tumor studies, and MDA-MB-231 cancer cell membrane coated NPs are used for the MDA-MB-231 tumor studies. Ten (10) mice per group per cell line are used for these studies.

### Dose Calculation

The concentration of CXCL12 in mouse plasma has been measured to be ˜500 pg/ml (Berahovich R D, Zabel B A, Lewen S, Walters M J, Ebsworth K, Wang Y, Jaen J C, Schall T J. Endothelial expression of CXCR7 and the regulation of systemic CXCL12 levels. Immunology. 2014; 141(1):111-22). Assuming the molecular weight of CXCL12 to be 10,000, the total mouse plasma volume of ˜2 mL, and using Avogadro's number this works out to ˜6×10¹⁰ molecules of CXCL12 in the mouse plasma. DU4475 cells have 16,640 CXCR4 receptors per cell and MDA-MB-231 cells have 6,833 CXCR4 receptors per cell (Avniel S, Arik Z, Maly A, Sagie A, Basst H B, Yahana M D, Weiss I D, Pal B, Wald O, Ad-El D, Fujii N, Arenzana-Seisdedos F, Jung S, Galun E, Gur E, Peled A. Involvement of the CXCL12/CXCR4 pathway in the recovery of skin following burns. J Invest Dermatol. 2006; 126(2):468-76). Assuming a very conservative estimate of achieving ˜3-10 CXCR4 receptors per NP, and assuming 1 mg of PLGA contains ˜1.5×10¹² NPs (2.5 pmol), a typical 100 μl injection of 0.1 mg/ml NP solution contains 1.5×10¹⁰ NPs or ˜4.5-15×10¹⁰ CXCR4 receptors. An injection of 100 μl of NPs should act as a nanosponge for most of the CXCL12 present in plasma and is the initial dosing concentration tested in vivo.

### Studies In Vivo

To determine the ability of the NPs to bind to activated circulating fibroblasts in mice, SCID mice are injected with fluorescently labeled activated NIH3T3 fibroblasts and 2 hours later are injected with the NPs (10 mice) using an optimum dose derived from the cell culture studies. Blood samples are withdrawn every 24 h over a period of 4 days to determine the binding of the NP to the circulating fibroblasts and determine changes in CXCL12 levels in the plasma of non-tumor bearing mice. Biodistribution studies at day 4 are also performed with these mice to determine the retention of the NPs in tissues and organs within the body in the absence of tumors.

The ability of the NPs to disrupt the formation of metastasis using cancer cells (10⁵) injected in the tail vein is determined. NPs are injected within 2 h and at 24 h and 72 h following cancer cell injection. Subsets of mice are euthanized at each time point for biodistribution studies, and to determine plasma CXCL12 levels using ELISA, and perform cytological analysis of blood smears to determine association between NPs and cancer cells, and CAFs. Analysis is also performed to determine if the NPs co-localize with the cancer cells in the inflated lungs, liver and lymph nodes. The remaining mice are euthanized at the end of five weeks to determine differences in metastatic burden, CAFs using α-SMA antibody immunostaining, and Col1 fiber patterns (using SHG microscopy) in metastatic nodules between the different treatment groups. Finally, the effect of the NPs on primary tumor growth and spontaneous metastasis is determined. Orthotopically implanted tumors are allowed to grow to ˜150 mm³ at which time mice are injected with the NPs every three days until the saline treatment tumor groups are ˜700 mm³. At these tumor volumes, axillary lymph node and lung metastasis are observed. Differences in tumor growth and in the metastatic burden in the lungs, liver, and axillary lymph nodes of these mice using hematoxylin and eosin (H&E) staining of fixed tissue sections are determined. The Col1 fiber patterns in primary and metastatic tumors is determined by SHG microscopy of these sections. Also α-SMA antibody immunostaining of CAFs in the tissue sections is performed. All tissue sections obtained from the experimental and spontaneous metastasis studies are routinely stained for proliferation (Ki-67), apoptosis (TUNEL assay), and endothelial cells (CD31).

### Statistical Analysis

The primary analysis uses multivariate nonparametric statistical tests to compare NP treated versus control groups in cultured cells and in vivo experiments. Based on power analysis, a sample size of 10 per group achieves 85% power when the average effect size is as low as 1.3. Mixed effects model are used to analyze tumor growth with random intercept to adjust for difference in baseline (t=0 days) tumor volume. The secondary analyses compares proliferation (Ki-67), apoptosis (TUNEL assay), CAFs (α-SMA), endothelial cells (CD31), Col1 fiber (SHG), metastatic burden and necrosis (H&E) measured from tumor sections between groups using t-test or Wilcoxon test based on data distribution. The tumor growth rate in the control group is about 40˜50 mm³/day. The tumor volume at the end of two weeks is about 600 to 750 mm³. The standard deviation is about 200 mm³. With 10 mice per group, at least 85% is used to detect effect size 1.42 or above in tumor volume reduction from targeted probe treated group with two-sided significance level alpha=0.05.

In one aspect, it is determined that DU4475 cancer cell membrane coated NPs are more effective at reducing CXCL12 levels than MDA-MB-231 cancer cell membrane coated NPs because of differences in CXCR4 receptor density on the membranes. CAFs are fewer in primary and metastatic tumors in the NP treated mice compared to control mice. NP injected mice also exhibit fewer Col1 fibers in primary and metastatic sites as well as fewer metastatic lesions. It is difficult to predict the effect on tumor growth but it is likely growth rate will decrease because of reduced CXCL12 in the plasma.

Fibroblast trafficking is critical in wound healing and it is important to consider the effect of these NPs on wound healing. One purpose of these NPs is to disrupt the CXCL12-CXCR4 axis, but not damage fibroblasts. Prior to the invention described herein, the role of CXCL12-CXCR4 in wound healing has not been closely investigated. There is some evidence that blocking the CXCL12-CXCR4 pathway may, in fact, improve skin recovery after burns (Avniel S, Arik Z, Maly A, Sagie A, Basst H B, Yahana M D, Weiss I D, Pal B, Wald O, Ad-El D, Fujii N, Arenzana-Seisdedos F, Jung S, Galun E, Gur E, Peled A. Involvement of the CXCL12/CXCR4 pathway in the recovery of skin following burns. J Invest Dermatol. 2006; 126(2):468-76). By using FAP-α antibody targeted NPs, the CXCL12-CXCR4 axis is disrupted specifically in activated fibroblasts, but hematopoietic stem cells are not affected where this axis is critical. The NP size of ˜70 nm should allow for a circulation time of ˜12 h and reasonable tumor delivery. Internalization of the NPs reduces the ability of the NPs to ‘sponge’ CXCL12, but there is at least a two-fold reduction of CXCL12. The purpose of these NPs is not solely to act as nanosponges for CXCL12 but to act as decoys to misdirect or distract cancer cells and stromal cells in the metastatic cascade. Focusing on the CXCR4-CXCL12 axis and its role in CAFs represent first steps in evaluating the functional effects of these NPs.

### Example 6: Cancer Cell Membrane-Coated Poly(Lactic-Co-Glycolic Acid) Nanoparticles

As described herein, biomimetic nanoparticles (NPs) combining synthetic and biological materials have flexibility and functionality. In this experiment, cancer cell membranes were coated onto poly(lactic-co-glycolic acid) (PLGA) NPs to translocate membrane anchored proteins onto NPs in a “right-side” out manner. As described herein, cancer cell membrane coated PLGA NPs disrupt cancer cell-stromal cell interactions and prime the immune system in cancer immunotherapy.

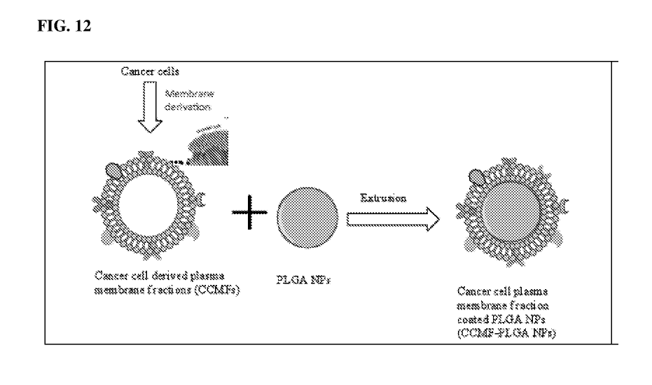

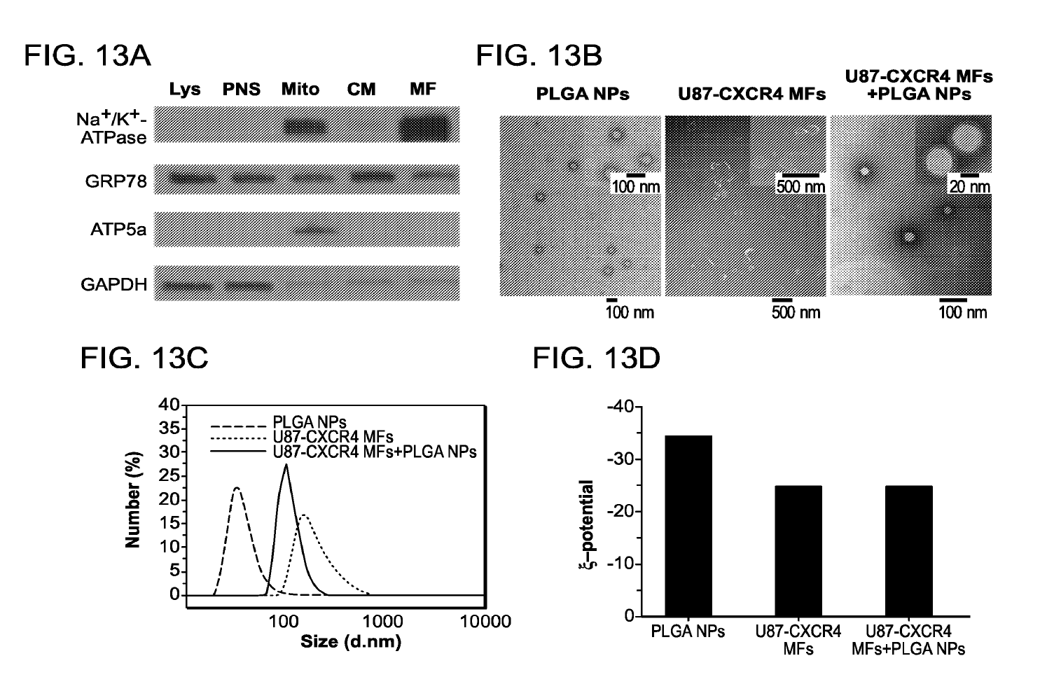

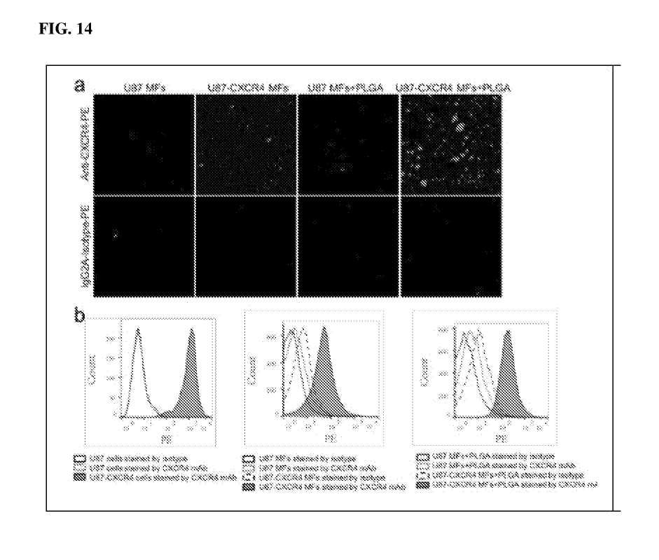

A schematic illustration of the preparation of the NPs is shown in FIG. 12. Characterization of the NPs is presented in FIG. 13A-FIG. 13D. Data presented in FIG. 14A-FIG. 14B demonstrate that the cancer cell membranes coated the NPs right side out. This is important for recognition of the NPs by stromal cells to disrupt cancer cell stromal cell interactions and for generating an immune response. As shown in FIG. 14A-FIG. 14B, NPs made from cells with high CXCR4 expression showed proportionately and significantly higher binding with CXCR4 antibodies, confirming that the receptor was intact following NP synthesis and recognized by the antibody. These results were replicated with CD44 binding on MDA-MB-231 triple negative breast cancer cells (TNBC) with high CD44 expression compared to BT474 cells with low CD44 expression.

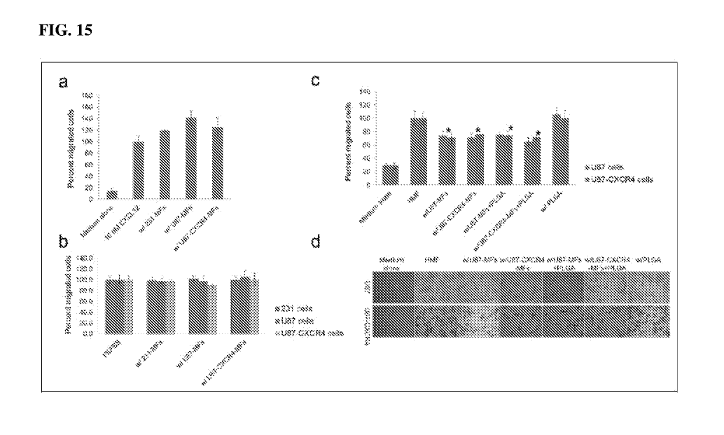

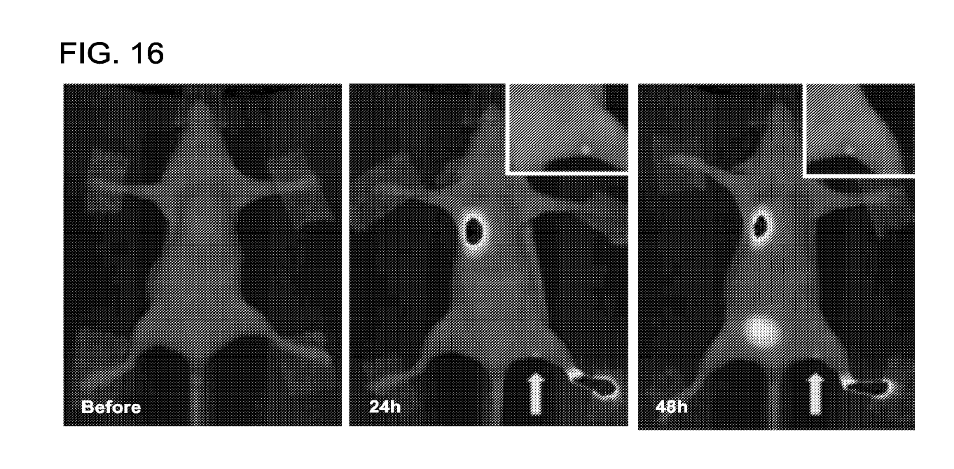

Next, the functionality of these NPs was evaluated and their ability to disrupt the interaction between cancer cells and human mammary fibroblasts (HMF) was determined. HMF cells were plated into each well of a 24-well companion plate (Corning) with 0.75 ml of cell suspension at density of 2×10⁴ cells/ml. After overnight incubation, medium was replenished with serum-free medium in the presence or absence of 40 μg of CCMFs or CCMF+PLGA NPs. A Falcon™ cell culture insert (8 μm, transparent PET membrane, Corning) was placed into every well and plated with 5×10⁴ U87 or U87-CXCR4 cell suspension in 0.5 ml of serum-free medium. The plate was then incubated further for one day. The cells inside the inserts were scraped off by cotton swabs, and the cells migrated to the bottom of the insert membrane were stained with 0.2% crystal violet (Sigma-Aldrich) in 20% methanol solution for cell counting under a microscope. The percent migration value was obtained by normalizing to the number of cells migrated to the medium alone. As shown in FIG. 15, incubation of HMFs with CCMFs or PLGA NPs coated with CCMF resulted in a significant reduction of invasion and migration of cancer cells across the insert membrane.

Next, it was determined whether CCMFs+PLGA NPs had the ability to localize in lymph nodes to generate an immune response. In initial studies, uptake of U87-CXCR4 CCMFs in the sciatic lymph node was observed within 24 h of footpad injection (FIG. 16), identifying a role for the NPs in providing cancer cell membranes to antigen presenting cells to induce a tumor-specific immune response.

### Example 7: Materials and Methods

The following materials and methods were utilized for Example 6.

### Cell Culture

U87, HMF, MDA-MB-231, and BT-474 cells were cultured in 10% fetal bovine serum (FBS, Sigma) supplemented MEM (Mediatech), DMEM (Mediatech), RPMI 1640 (Sigma), and ATCC 46-X (ATCC) media, respectively. U87-CXCR4 was maintained in DMEM medium supplemented with 15% FBS, 1 μg/ml puromycin (Sigma-Aldrich), 300 μg/ml G418 (Mediatech). Cells were maintained at 37° C. in a humidified atmosphere containing 5% CO₂.

### Preparation of Cancer Cell Membrane Fractions (CCMFs)

CCMFs were harvested from source cancer cells. Briefly, cells were grown in 150-mm peri dishes to full confluency (four peri dishes for each cell line), and detached with 10 mM ethylenediaminetetraacetic acid (EDTA, Sigma-Aldrich) in 1× phosphate buffered saline (PBS, pH 7.4, Sigma-Aldrich) to prepare CCMFs.

### Preparation of Cancer Cell Membrane Fraction-Coated PLGA NPs (CCMF+PLGA NPs)

CCMFs were extruded through a 400-nm polycarbonate porous membrane (Avanti Polar Lipids, Inc) to harvest cancer cell membrane vesicles. Poly(DL-lactic-co-glycolic acid) (PLGA) NPs were prepared using a nanoprecipitation method. Cancer cell membrane vesicles and PLGA NPs were mixed in a certain ratio and physically extruded through a 400-nm polycarbonate porous membrane for eleven passes to obtain CCMF+PLGA NPs.

### Immunoblot Assay

Various subcellular fractions were lysed in radioimmune precipitation (RIPA, Sigma-Aldrich) buffer and measured by a BCA assay (Pierce) for protein assay. Samples with the same amount of protein loading were fractionated by SDS-PAGE, and transferred to a nitrocellulose membrane. A membrane fraction antibody cocktail (ab140365, Abcam), consisting of antibodies targeting anti-sodium potassium ATPase for plasma membrane, GRP78 for endoplasmic reticulum, ATPSA for mitochondria, and GAPDH for cytosol, was used at 1:250 dilution for membrane immunoblotting. Horseradish peroxidase-conjugated secondary antibody cocktail (ab140365, Abcam) was used at 1:2500 dilution, and the signal was developed using ECL Plus reagents (Thermo Scientific). Membranes were stripped and reprobed with anti-CXCR4 antibody (Prosci) and anti-CD44 antibody (clone 8E2, Cell Signaling).

### Transmission Electron Microscopy (TEM) and Dynamic Laser Scattering (DLS) Measurements

Carbon-coated 400 square mesh copper grids (CF400-Cu, Electron Microscopy Sciences) were first glow discharged, and floated onto a drop of sample solution for 2 min. Subsequently, grids were consecutively negatively stained with two drops of 1% phosphotungstic acid (PTA, Sigma-Aldrich) at pH 7.0 for 30 s. Excess solution was wicked away by filter paper between each staining process. TEM imaging was carried out on a Philips/FBI BioTwin CM120 microscopy at 80 kV. A Malvern Zetasizer Nano ZS90 was used to detect the information of particle size and zeta-potential of NPs.

### Confocal Microscopy

CCMFs or CCMF+PLGA NPs with 100 μg of protein was suspended in 100 μl of 1× PBS supplemented with 1% BSA and then added with 20 μl of Phycoerythrin (PE)-conjugated anti-human CXCR4 mouse monoclonal antibody (clone 12G5, R&D Systems) or 20 μl of APC-conjugated anti-human CD44 mouse monoclonal antibody (clone G44-26, BD Pharmingen™). PE-conjugated mouse IgG2A isotype (clone 20102, R&D Systems) or APC-conjugated mouse IgG2b× isotype (clone 27-25, BD Pharmingen™) was used as control. The mixture was kept at RT for 1 h under occasionally stirring, washed with 1×PBS for twice and pelleted by centrifugation at 20,000×g for 30 min. The resulting pellet was resuspended in 100 μl of 1×PBS. A drop of sample suspension was placed onto a Fisherbrand® microscope cover glass (22 mm×60 mm, Fisher Scientific), and imaged by a laser scanning confocal microscope (Zeiss LSM 510-Meta, Carl Zeiss Microscopy GmbH). The laser wavelength was set at 561 nm or 633 nm, and the receiving PMT channel was set at 572-625 nm or 650-700 nm for imaging CXCR4 or CD44 proteins, respectively. All the images presented the same group were obtained under identical microscope settings.

### Flow Cytometry

Cells were detached using 1× non-enzymatic cell dissociation solution (Sigma), washed and suspended in 1×PBS supplemented with 1% BSA. To examine the expression levels of CXCR4 on U87 and U87-CXCR4 cells, 1×10⁶ of live cells were stained with 20 μl of Phycoerythrin (PE)-conjugated anti-human CXCR4 mouse monoclonal antibody at 4° C. for 1 h. For the CD44 levels on MDA-MB-231 and BT-474 cells, 20 μl of APC-conjugated anti-human CD44 mouse monoclonal antibody was used. For the sample preparation towards MFs and MF+PLGA NPs, the procedure was the same as described in Confocal microscopy section. Flow cytometry measurement was conducted on a FACS Calibur (BD Bioscience) and ten thousand events were collected for each measurement and analyzed by FlowJo software (FLOWJO).

### Integrity Study on CCMF+PLGA NPs

To investigate the integrity of CCMF+PLGA NPs and verify the MF coating on PLGA NP cores, CCMF+PLGA NPs were doubly labeled with a fluorescent antibody towards MF coating and a DiD dye in the core. Briefly, U87-CXCR4 MFs+PLGA-DiD NPs were stained by Phycoerythrin (PE)-conjugated anti-human CXCR4 mouse monoclonal antibody according to the procedure as described in confocal microscopy section. U87-CXCR4 MFs without PLGA core were used as control. MDA-MB-231 MFs+PLGA-DiD NPs were sequentially stained with 2 μg/ml of anti-CD44 monoclonal antibody (clone MEM263, Sigma) at RT for 1 h, and secondarily stained by 2 μg/ml of Alexa 488 labeled goat anti-mouse secondary antibody (Life Technologies) at RT for 30 min MDA-MB-231 MFs receiving the same procedure of staining were taken as control. PE fluorescence was recorded according to settings as described in “confocal microscopy” section. Alexa 488 fluorescence from the NP shell was acquired by the microscope with excitation at 488 nm and the emission filter of LP505, and DiD signals from the NP core were obtained with excitation at 633 nm and emission PMT channel of 650-700 nm. All the images in the same comparison group were acquired under identical experimental settings.

### Cell Migration Assay

HMF cells were plated into each well of a 24-well companion plate (Corning) with 0.75 ml of cell suspension at density of 2×10⁴ cells/ml. After overnight incubation, medium was replenished with serum-free medium in the presence or absence of 40 μg of CCMFs or CCMF+PLGA NPs. A Falcon™ cell culture insert (8 μm, transparent PET membrane, Corning) was placed into every well and plated with 5×10⁴ U87 or U87-CXCR4 cell suspension in 0.5 ml of serum-free medium. The plate was then incubated further for one day. The cells inside the inserts were scraped off by cotton swabs, and the cells migrated to the bottom of the insert membrane were stained with 0.2% crystal violet (Sigma-Aldrich) in 20% methanol solution for cell counting under a microscope. The percent migration value was obtained by normalizing to the number of cells migrated to the medium alone.

### Statistical Analysis

Data were expressed as mean±SD from at least three samples or animals. Statistical analysis was performed with one-sided student t-test (Microsoft Excel), assuming unequal variance. Values of P=0.05 were considered significant, unless otherwise stated.

### EQUIVALENTS

Those skilled in the art will recognize, or be able to ascertain using no more than routine experimentation, many equivalents to the specific embodiments of the invention described herein. Such equivalents are intended to be encompassed by the following claims.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A composition comprising a nanoparticle, wherein the nanoparticle surface is encapsulated with one or more plasma membrane-associated components, and wherein the plasma membrane is derived from a cancer cell.

2. The composition of claim 1, wherein the plasma membrane-associated component comprises a lipid, a protein, or a carbohydrate.

3. The composition of claim 1, wherein the plasma membrane-associated component is retained in a native conformation within the plasma membrane on the surface of the nanoparticle.

4. The composition of claim 1, wherein the plasma membrane-associated component is present in the right-side-out orientation on the surface of the nanoparticle.

5. The composition of claim 1, wherein said plasma membrane comprises a bilayer.

6. The composition of claim 1, wherein said nanoparticle is negatively charged.

7. The composition of claim 1, wherein said nanoparticle is 40-150 nm in size.

8. The composition of claim 1, wherein the nanoparticle comprises a polymeric nanoparticle.

9. The composition of claim 1, wherein the nanoparticle comprises a polylactic-co-glycolic acid (PLGA) polymeric nanoparticle.

10. The composition of claim 1, wherein the plasma membrane is about 5 nm thick.

11. The composition of claim 1, further comprising a detectable label.

12-17. (canceled)

18. The composition of claim 1, wherein said plasma membrane comprises CXCR4.

19. The composition of claim 1, wherein said plasma membrane comprises a protein selected from the group comprising CEA (carcinoembryonic antigen), HER2 (human epidermal growth factor receptor 2), CD44 (cluster of differentiation 44) and PSMA (prostate-specific membrane antigen).

20. (canceled)

21. The composition of claim 1, wherein the cancer cell comprises a breast cancer cell.

22. A method of treating cancer comprising:
isolating a cancer cell from a subject;
administering to the subject a composition comprising a nanoparticle, wherein the nanoparticle surface is encapsulated with one or more plasma membrane-associated components, and wherein the plasma membrane is derived from the cancer cell; and
activating an immune response against the cancer cell in the subject, thereby treating the cancer.

23. The composition of claim 22, wherein the cancer is selected from the group consisting of breast cancer, skin cancer, lung cancer, brain cancer, pancreatic cancer, esophageal cancer, stomach cancer, liver cancer, kidney cancer, colorectal cancer, intestinal cancer, bladder cancer, prostate cancer, ovarian cancer, uterine cancer, testicular cancer, sarcoma, lymphoma, leukemia, retinoblastoma, oral cancer, bone cancer, neoplasia, dysplasia, and glioma.

24. The method of claim 23, wherein the composition further comprises a drug or pharmaceutical composition.

25. (canceled)

26. A method of disrupting cancer cell-stromal cell signaling in a subject comprising:
isolating a cancer cell from a subject;
administering to the subject a composition comprising a nanoparticle, wherein the nanoparticle surface is encapsulated with one or more plasma membrane-associated components, and wherein the plasma membrane is derived from the cancer cell, thereby disrupting cancer cell-stromal cell signaling in the subject,
or,
isolating a cancer cell from a subject;
administering to the subject a composition comprising a nanoparticle, wherein the nanoparticle surface is encapsulated with one or more plasma membrane-associated components, and wherein the plasma membrane is derived from the cancer cell, wherein the composition further comprises a detectable label; and
identifying the detectable label, thereby detecting a cancer cell-stromal cell interaction.

27-30. (canceled)

31. A method of preparing a composition comprising a nanoparticle, wherein the nanoparticle surface is encapsulated with one or more plasma membrane-associated components, and wherein the plasma membrane is derived from a cancer cell, the method comprising:
isolating a cancer cell;
fractionating the cancer cell into one or more plasma membrane-derived vesicles;
synthesizing polymeric nanoparticles; and
fusing the plasma membrane-derived vesicle with the nanoparticle, thereby preparing a composition comprising a nanoparticle.

32. The method of claim 31, wherein fractionating the cancer cell into one or more plasma membrane-derived vesicles comprises sequentially homogenizing and gradient-density centrifuging the cancer cell.

33-35. (canceled)
